SEQUENCE LISTING (106 sequences)

SEQ ID NO: 1 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 428 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 3 - protein, 330 aa, synthetic construct [sequence omitted]

SEQ ID NO: 4 - protein, 758 aa, synthetic construct
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 142 aa, Homo sapiens
AEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQ

SEQ ID NO: 6 - protein, 92 aa, Homo sapiens
ASMTASPPGSLRASDWVILNCSFSRPDRPASVHWFRNRGQGRVPVRESPHHHLAESFLFLPQVSPMDSGPWGCILTYRDGFNVSIMYNLTVL

SEQ ID NO: 7 - protein, 89 aa, Homo sapiens
EPPTPLTVYAGAGSRVGLPCRLPAGVGTRSFLTAKWTPPGGGPDLLVTGDNGDFTLRLEDVSQAQAGTYTCHIHLQEQQLNATVTLAII

SEQ ID NO: 8 - protein, 80 aa, Homo sapiens
TVTPKSFGSPGSLGKLLCEVTPVSGQERFVWSSLDTPSQRSFSGPWLEAQEAQLLSQPWQCQLYQGERLLGAAVYFTELS

SEQ ID NO: 9 - protein, 103 aa, synthetic construct
DIQMTQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTK

SEQ ID NO: 10 - protein, 120 aa, synthetic construct
QVQLKQSDDELVKPGASVKISCKVSGYTFTDHTIHWMKQRPEQGLEWIGYIYPRDGSTKYNEKFKGKATLTADKSSSTAYMQLNSLTSEDSAVYFCARRRSYYGSSFYFDVWGTGTTVSV

SEQ ID NO: 11 - protein, 100 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGG

SEQ ID NO: 12 - protein, 119 aa, synthetic construct
VQLQESGAEVKKPGSSVKVSCKASGGPFRSYAISWVRQAPGQGPEWMGGIIPIFGTTKYAPKFQGRVTITADDFAGTVYMELSSLRSEDTAMYYCAKHMGYQVRETMDVWGKGTTVTVS

SEQ ID NO: 13 - protein, 103 aa, synthetic construct
DIQMTQVTSSLSASLGDRVTISCRASQDISNYLNWYQQKPDGSVKLLIYYTSRLHSGVPSRFSGSGSGTDYSLTISNLEQEDIATYFCQQGNTLPRTFGGGTK

SEQ ID NO: 14 - protein, 103 aa, synthetic construct
DIVMTQSPASLSVSVGETVTITCRASENIYSNLAWYQQKQGKSPQLLVYAATNLADGVPSRFSGSGSGTQYSLKINSLQSEDFGSYYCQHFWGTPRTFGGGTK

SEQ ID NO: 15 - protein, 103 aa, synthetic construct
IVITQSPAIMSASLGERVTMTCTVTSSISSSYLHWYQQKPGSSPKLWIYSTSNLASGVPTRFSGSGSGTSYSLTVSSMEAEDAATYYCHQYHRSPFTFGSGTK

SEQ ID NO: 16 - protein, 116 aa, synthetic construct
LVESGAEVKKPGSSVKVSCKASGGPFRSYAISWVRQAPGQGPEWMGGIIPIFGTTKYAPKFQGRVTITADDFAGTVYMELSSLRSEDTAMYYCAKHMGYLVRETMDVWGKGTTVTV

SEQ ID NO: 17 - protein, 104 aa, synthetic construct
DIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGG

SEQ ID NO: 18 - protein, 117 aa, synthetic construct
QVQLKQSDAELVKPGASVKISCKVSGYTFTDHTIHWMKQRPEQGLEWIGYIYPRDGSTKYNEKFKGKATLTADKSSSTAYMQLNSLTSEDSAVCFCARTGSYAMDYWGQGTSVTVSS

SEQ ID NO: 19 - protein, 103 aa, synthetic construct
EIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPYTFGGGTK

SEQ ID NO: 20 - protein, 122 aa, synthetic construct
QVQLQQSGAELVKPGASVKLSCKASGYTFTSYWMHWVKQRPGRGLEWIGRIDPNSGGTKYNEKFKSKATLTVDKPSSTAYMQLSSLTSEDSAVYYCARGKLAGYYGYYFDYWGQGTTLTVSS

SEQ ID NO: 21 - protein, 102 aa, synthetic construct
DIVMTQSHKFMSTSVGDRISITCKASQDVGTAVAWYQQKPGQSPKLLIFWTSTRHTGVPDRFAGSRSGTDFTLTISNVQSEDLADYFCQQYSSYPYTFGGGT

SEQ ID NO: 22 - protein, 110 aa, synthetic construct
DIVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKXE

SEQ ID NO: 23 - protein, 103 aa, synthetic construct
DIQMTQSPASLSVSVGETVTITCRASENIYSNLAWYQQKQGKSPQLLVYAATNLADGVPSRFSGSGSGTQYSLKINSLQSEDFGSYYCQHFWGTPYTFGGGTK

SEQ ID NO: 24 - protein, 108 aa, synthetic construct
DIVMTQTPLTLSVTIGQPASISCKSSQSLLDSDGKTYLNWLLQRPGQSPKRLIYLVSKLDSGVPDRFTGSGSGTEFTLKISRVEAEDLGVYYCWQGTHFPRTFGGGTK

SEQ ID NO: 25 - protein, 124 aa, synthetic construct
QVQLQQSGGELVMHGASVKLSCKDSGYAFSSYWMLWVKQRPGQDLEWMGDIDLSDIYTKYNQKFKGKSTLTVDKSSSTAYMQVSSMTCEDYAVYYCARGGYWYGISYGSAMGYWGQGTSVSVSS

SEQ ID NO: 26 - protein, 108 aa, synthetic construct
DIVMTQTPLSLPVSLGDHASISCRSSQSIVHSNGNTYLEWYLQKPGQSSSSNFNRCTFRFCDDPDRFSGSGSGTDFTLKISRVEAEDLGVYYCFQGSHVPWTFGGGTK

SEQ ID NO: 27 - protein, 104 aa, synthetic construct
SVKVSCKASGGPFRSYAISWVRQAPGQGPEWMGGIIPIFGTTKYAPKFQGRVTITADDFAGTVYMELSSLRSEDTAMYYCAKHMGYQVRETMDVWGKGTTVTVS

SEQ ID NO: 28 - protein, 103 aa, synthetic construct
DIVMTQSPSSLSVSVGETVTITCRASENIYSNLAWYQQKQGKSPQLLVYAATNLADGVPSRFSGSGSGTQYSLKINSLQSEDFGSYYCQHFWGTPWTFGGGTK

SEQ ID NO: 29 - protein, 107 aa, synthetic construct
DIVMTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTK

SEQ ID NO: 30 - protein, 119 aa, synthetic construct
VQLQESGAEVKKPGSSVKVSCKASGGPFRSYAISWVRQAPGQGLEWMGGIIPICGTTKYAQKFQGRVTITADDFSGTVYMELSSLRSEDSAMYYCAKHMGYQVSETMDVWGKGTAVTVS

SEQ ID NO: 31 - protein, 101 aa, synthetic construct
IVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTK

SEQ ID NO: 32 - protein, 114 aa, synthetic construct
QVQLKQSGAELVKPGASVKLSCKASGYTFTSYWMHWVKQRPGQGLGWIGMIHPNSGSTNYNEKFKSKATLTVDKSSSTAYMQLSSLTSEDSAVYYCAISPYFDYWGQGTTLTVS

SEQ ID NO: 33 - protein, 108 aa, synthetic construct
DIVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTK

SEQ ID NO: 34 - protein, 108 aa, synthetic construct
DIVMTQTPLTLSVTIGQPASISCKSSQSLLDSDGKTYLNWLLQRPGQSPKRLIYLVSKLDSGVPDRFTGSGSRTDFTLKISRVEAEDLGVYYCWQGTHFPQTFGGGTK

SEQ ID NO: 35 - protein, 6 aa, synthetic construct
QDISNY

SEQ ID NO: 36 - protein, 9 aa, synthetic construct
QQGNTLPWT

SEQ ID NO: 37 - protein, 8 aa, synthetic construct
GYTFTDHT

SEQ ID NO: 38 - protein, 8 aa, synthetic construct
IYPRDGST

SEQ ID NO: 39 - protein, 15 aa, synthetic construct
ARRRSYYGSSFYFDV

SEQ ID NO: 40 - protein, 9 aa, synthetic construct
QQGNTLPYT

SEQ ID NO: 41 - protein, 8 aa, synthetic construct
GGPFRSYA

SEQ ID NO: 42 - protein, 8 aa, synthetic construct
IIPIFGTT

SEQ ID NO: 43 - protein, 14 aa, synthetic construct
AKHMGYQVRETMDV

SEQ ID NO: 44 - protein, 9 aa, synthetic construct
QQGNTLPRT

SEQ ID NO: 45 - protein, 6 aa, synthetic construct
ENIYSN

SEQ ID NO: 46 - protein, 9 aa, synthetic construct
QHFWGTPRT

SEQ ID NO: 47 - protein, 7 aa, synthetic construct
SSISSSY

SEQ ID NO: 48 - protein, 9 aa, synthetic construct
HQYHRSPFT

SEQ ID NO: 49 - protein, 14 aa, synthetic construct
AKHMGYLVRETMDV

SEQ ID NO: 50 - protein, 10 aa, synthetic construct
QSVDYDGDSY

SEQ ID NO: 51 - protein, 9 aa, synthetic construct
QQSNEDPYT

SEQ ID NO: 52 - protein, 10 aa, synthetic construct
ARTGSYAMDY

SEQ ID NO: 53 - protein, 6 aa, synthetic construct
QDVSTA

SEQ ID NO: 54 - protein, 9 aa, synthetic construct
QQHYSTPYT

SEQ ID NO: 55 - protein, 8 aa, synthetic construct
IDPNSGGT

SEQ ID NO: 56 - protein, 15 aa, synthetic construct
ARGKLAGYYGYYFDY

SEQ ID NO: 57 - protein, 6 aa, synthetic construct
QDVGTA

SEQ ID NO: 58 - protein, 9 aa, synthetic construct
QQYSSYPYT

SEQ ID NO: 59 - protein, 11 aa, synthetic construct
QSLVHSNGNTY

SEQ ID NO: 60 - protein, 9 aa, synthetic construct
SQSTHVPYT

SEQ ID NO: 61 - protein, 9 aa, synthetic construct
QHFWGTPYT

SEQ ID NO: 62 - protein, 11 aa, synthetic construct
QSLLDSDGKTY

SEQ ID NO: 63 - protein, 9 aa, synthetic construct
WQGTHFPRT

SEQ ID NO: 64 - protein, 8 aa, synthetic construct
GYAFSSYW

SEQ ID NO: 65 - protein, 8 aa, synthetic construct
IDLSDIYT

SEQ ID NO: 66 - protein, 17 aa, synthetic construct
ARGGYWYGISYGSAMGY

SEQ ID NO: 67 - protein, 11 aa, synthetic construct
QSIVHSNGNTY

SEQ ID NO: 68 - protein, 9 aa, synthetic construct
FQGSHVPWT

SEQ ID NO: 69 - protein, 9 aa, synthetic construct
QHFWGTPWT

SEQ ID NO: 70 - protein, 10 aa, synthetic construct
ESVDNYGISF

SEQ ID NO: 71 - protein, 9 aa, synthetic construct
QQSKEVPWT

SEQ ID NO: 72 - protein, 8 aa, synthetic construct
IIPICGTT

SEQ ID NO: 73 - protein, 14 aa, synthetic construct
AKHMGYQVSETMDV

SEQ ID NO: 74 - protein, 5 aa, synthetic construct
SSVSY

SEQ ID NO: 75 - protein, 9 aa, synthetic construct
QQWSSYPLT

SEQ ID NO: 76 - protein, 8 aa, synthetic construct
GYTFTSYW

SEQ ID NO: 77 - protein, 8 aa, synthetic construct
IHPNSGST

SEQ ID NO: 78 - protein, 8 aa, synthetic construct
AISPYFDY

SEQ ID NO: 79 - protein, 9 aa, synthetic construct
SQSTHVPWT

SEQ ID NO: 80 - protein, 9 aa, synthetic construct
WQGTHFPQT

SEQ ID NO: 81 - DNA, 310 bp, synthetic construct
  source 1..310: mol_type = other DNA
gatatccagatgacacagtctacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagc

SEQ ID NO: 82 - DNA, 360 bp, synthetic construct
  source 1..360: mol_type = other DNA
caggtgcagctgaagcagtctgacgatgagttggtgaaacctggagcttcagtgaagatatcctgcaaggtttctggctacaccttcactgaccatactattcactggatgaagcagaggcctgaacagggcctggaatggattggatatatttatcctagagatggtagtactaagtacaatgagaagttcaagggcaaggccacattgactgcagacaaatcctccagcacagcctacatgcagctcaacagcctgacatctgaggactctgcagtctatttctgtgcaagaagacggagttactacggtagtagcttttacttcgatgtctggggcacagggaccacggtctccgtc

SEQ ID NO: 83 - DNA, 299 bp, synthetic construct
  source 1..299: mol_type = other DNA
gatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtacacgttcggagg

SEQ ID NO: 84 - DNA, 356 bp, synthetic construct
  source 1..356: mol_type = other DNA
gtgcagctgcaggagtctggggctgaggtgaagaagcctgggtcctcggtgaaagtctcttgcaaggcttctggaggccccttccgcagctatgctatcagctgggtgcgacaggcccctggacaagggcctgagtggatgggagggatcatccctatttttggtacaacaaaatacgcaccgaagttccagggcagagtcacgattaccgcggacgatttcgcgggcacagtttacatggagctgagcagcctgcgatctgaggacacggccatgtactactgtgcgaaacatatggggtaccaggtgcgcgaaactatggacgtctggggcaaagggaccacggtcaccgtctc

SEQ ID NO: 85 - DNA, 309 bp, synthetic construct
  source 1..309: mol_type = other DNA
gatatccagatgacccaggttacatcctccctgtctgcctctctgggagacagagtcaccatcagttgcagggcaagtcaggacattagcaattatttaaactggtatcagcagaaaccagatggatctgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttcctcggacgttcggtggaggcaccaag

SEQ ID NO: 86 - DNA, 309 bp, synthetic construct
  source 1..309: mol_type = other DNA
gacattgtgatgacccagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactcctcggacgttcggtggaggcaccaag

SEQ ID NO: 87 - DNA, 309 bp, synthetic construct
  source 1..309: mol_type = other DNA
attgtgatcacccagtctccagcaatcatgtctgcatctctaggggaacgggtcaccatgacctgcactgtcacctcaagtattagttccagttacttgcactggtaccagcagaagccaggatcctcccccaaactctggatttatagcacatccaacctggcttctggagtcccaactcgcttcagtggcagtgggtctgggacctcttactctctcacagtcagcagcatggaggctgaagatgctgccacttattactgccaccagtatcatcgttccccattcacgttcggctcggggacaaag

SEQ ID NO: 88 - DNA, 356 bp, synthetic construct
  source 1..356: mol_type = other DNA
gtgcagctggtggagtctggggctgaggtgaagaagcctgggtcctcggtgaaagtctcttgcaaggcttctggaggccccttccgcagctatgctatcagctgggtgcgacaggcccctggacaagggcctgagtggatgggagggatcatccctatttttggtacaacaaaatacgcaccgaagttccagggcagagtcacgattaccgcggacgatttcgcgggcacagtttacatggagctgagcagcctgcgatctgaggacacggccatgtactactgtgcgaaacatatggggtaccaggtgcgcgaaactatggacgtctggggcaaagggaccacggtcaccgtctc

SEQ ID NO: 89 - DNA, 311 bp, synthetic construct
  source 1..311: mol_type = other DNA
gacattgtgatgacccagtctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggagg

SEQ ID NO: 90 - DNA, 352 bp, synthetic construct
  source 1..352: mol_type = other DNA
caggtgcagctgaagcagtctgacgctgagttggtgaaacctggagcttcagtgaagatatcctgcaaggtttctggctacaccttcactgaccatactattcactggatgaagcagaggcctgaacagggcctggaatggattggatatatttatcctagagatggtagtactaagtacaatgagaagttcaagggcaaggccacattgactgcagacaaatcctccagcacagcctacatgcagctcaacagcctgacatctgaggactctgcagtctgtttctgtgcaagaactggtagctatgctatggactactggggtcagggaacctcagtcaccgtctcctcag

SEQ ID NO: 91 - DNA, 309 bp, synthetic construct
  source 1..309: mol_type = other DNA
gaaattgtgatgacccagtctcacaaattcatgtccacatcagtaggagacagggtcagcatcacctgcaaggccagtcaggatgtgagtactgctgtagcctggtatcaacagaaaccaggacaatctcctaaactactgatttactcggcatcctaccggtacactggagtccctgatcgcttcactggcagtggatctgggacggatttcactttcaccatcagcagtgtgcaggctgaagacctggcagtttattactgtcagcaacattatagtactccgtacacgttcggaggggggaccaag

SEQ ID NO: 92 - DNA, 367 bp, synthetic construct
  source 1..367: mol_type = other DNA
caggtgcagctgcagcagtctggggctgagcttgtgaagcctggggcttcagtgaagctgtcctgcaaggcttctggctacaccttcaccagctactggatgcactgggtgaagcagaggcctggacgaggccttgagtggattggaaggattgatcctaatagtggtggtactaagtacaatgagaagttcaagagcaaggccacactgactgtagacaaaccctccagcacagcctacatgcagctcagcagcctgacatctgaggactctgcggtctattattgtgcaagaggaaagcttgctggttactacgggtactactttgactactggggccaaggcaccactctcacagtctcctcag

SEQ ID NO: 93 - DNA, 308 bp, synthetic construct
  source 1..308: mol_type = other DNA
gacattgtgatgacccagtctcacaaattcatgtccacatcagtaggagacaggatcagcatcacctgcaaggccagtcaggatgtgggtactgctgtagcctggtatcaacagaaaccaggacaatctcctaaactactgattttctggacatccacccggcacactggagtccctgatcgcttcgcaggcagtagatctgggacagatttcactctcaccattagcaatgtgcagtctgaagacttggcagattatttctgtcagcaatatagcagctatccgtacacgttcggaggggggaccaa

SEQ ID NO: 94 - DNA, 331 bp, synthetic construct
  source 1..331: mol_type = other DNA
gatattgtgatgacccagactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtacacgttcggaggggggaccaagcnggaaa

SEQ ID NO: 95 - DNA, 305 bp, synthetic construct
  source 1..305: mol_type = other DNA
gacatccagatgacccagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactccgtacacgttcggaggggggac

SEQ ID NO: 96 - DNA, 324 bp, synthetic construct
  source 1..324: mol_type = other DNA
gatattgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtcaaaactggactctggagtccctgacaggttcactggcagtggatcagggacagaattcacactgaaaatcagcagagtggaggctgaggatttgggagtttattattgctggcaaggtacacattttcctcggacgttcggtggaggcaccaag

SEQ ID NO: 97 - DNA, 373 bp, synthetic construct
  source 1..373: mol_type = other DNA
caggtgcagctgcagcagtctgggggtgagcttgtgatgcatggggcttcagtgaagctgtcctgcaaggattctggctacgccttctccagctactggatgctctgggtgaagcagaggcctggacaagaccttgaatggatgggagacattgatctttctgatatttatactaaatacaatcaaaagttcaagggcaagtccacattgactgtagacaaatcctccagcacagcatacatgcaggtcagcagcatgacatgtgaggactatgcggtctattactgtgcaagagggggttattggtacggtattagctacggaagtgctatggggtactggggtcaaggaacctcagtctccgtctcctcag

SEQ ID NO: 98 - DNA, 324 bp, synthetic construct
  source 1..324: mol_type = other DNA
gatattgtgatgacccaaactccactctccctgcctgtcagtcttggagatcacgcctccatctcttgcagatctagtcagagcattgtacatagtaatggaaacacctatttagaatggtacctgcagaaaccaggccagtcttctagtagcaacttcaaccggtgtacattccgattttgtgatgacccagacaggttcagtggcagtggatcagggacagatttcacactcaagatcagcagagtggaggctgaggatctgggagtttattactgctttcaaggttcacatgttccgtggacgttcggtggaggcaccaag

SEQ ID NO: 99 - DNA, 311 bp, synthetic construct
  source 1..311: mol_type = other DNA
tcggtgaaagtctcttgcaaggcttctggaggccccttccgcagctatgctatcagctgggtgcgacaggcccctggacaagggcctgagtggatgggagggatcatccctatttttggtacaacaaaatacgcaccgaagttccagggcagagtcacgattaccgcggacgatttcgcgggcacagtttacatggagctgagcagcctgcgatctgaggacacggccatgtactactgtgcgaaacatatggggtaccaggtgcgcgaaactatggacgtctggggcaaagggaccacggtcaccgtctc

SEQ ID NO: 100 - DNA, 309 bp, synthetic construct
  source 1..309: mol_type = other DNA
gacattgtgatgacccagtctccatcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactccgtggacgttcggtggaggcaccaag

SEQ ID NO: 101 - DNA, 321 bp, synthetic construct
  source 1..321: mol_type = other DNA
gacattgtgatgacccaatctccagcttctttggctgtgtctctagggcagagggccaccatctcctgcagagccagcgaaagtgttgataattatggcattagttttatgaactggttccaacagaaaccaggacagccacccaaactcctcatctatgctgcatccaaccaaggatccggggtccctgccaggtttagtggcagtgggtctgggacagacttcagcctcaacatccatcctatggaggaggatgatactgcaatgtatttctgtcagcaaagtaaggaggttccgtggacgttcggtggaggcaccaag

SEQ ID NO: 102 - DNA, 356 bp, synthetic construct
  source 1..356: mol_type = other DNA
gtgcagctgcaggagtctggggctgaggtgaagaagcctgggtcctcggtgaaagtgtcttgcaaggcttctggaggccccttccgcagctatgctatcagctgggtgagacaggcccctggacaaggtcttgagtggatgggagggatcatccctatttgtggtacaacaaaatacgctcagaagttccagggcagagtcacgattaccgcggacgatttctcgggcacagtttacatggagctgagcagcctgcgatctgaggactcggccatgtattactgtgcgaaacatatggggtaccaggtgagcgaaactatggacgtctggggcaaagggaccgcggtcaccgtctc

SEQ ID NO: 103 - DNA, 303 bp, synthetic construct
  source 1..303: mol_type = other DNA
attgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccactcacgttcggtgctgggaccaag

SEQ ID NO: 104 - DNA, 341 bp, synthetic construct
  source 1..341: mol_type = other DNA
caggtgcaactgaagcagtctggggctgagctggtaaagcctggggcttcagtgaagttgtcctgcaaggcttctggctacactttcaccagctactggatgcactgggtgaagcagaggcctggacaaggccttgggtggattggaatgattcatcctaatagtggtagtactaactacaatgagaagttcaagagcaaggccacactgactgtagacaaatcctccagcacagcctacatgcaactcagcagcctgacatctgaggactctgcggtctattactgtgcaatatctccttactttgactactggggccaaggcaccactctcacagtctc

SEQ ID NO: 105 - DNA, 324 bp, synthetic construct
  source 1..324: mol_type = other DNA
gatattgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcacggacagatttcacactgaaaatcagcagagtggaggctgaggatttgggagtttattattgctggcaaggtacacattttcctcagacgttcggtggaggcaccaag

SEQ ID NO: 106 - DNA, 324 bp, synthetic construct
  source 1..324: mol_type = other DNA
gatattgtgatgacccagactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaag

(19) United States
(12) Patent Application Publication
(10) Pub. No.: US 2026/0265372 A1
(43) Pub. Date: Sep. 10, 2026

(54) LAG-3 INHIBITION FOR ENHANCED ANTIVIRAL IMMUNE RESPONSE

(71) Applicant: Technion Research and Development Foundation Limited, Haifa (IL)

(72) Inventors: Yotam BAR-ON, Haifa (IL); Dina KHATEEB, Haifa (IL)

(21) Appl. No.: 19/164,874

(22) PCT Filed: Mar. 14, 2024
(86) PCT No.: PCT/IL2024/050277

§ 371 Date: Sep. 12, 2025

Related U.S. Application Data
(60) Provisional application No. 63/490,530, filed on Mar. 16, 2023.

(51) Int. Cl. C07K 16/28; A61P 31/12

(52) U.S. Cl. CPC C07K 16/2803; A61P 31/12; C07K 2317/565; C07K 2317/76; C07K 2317/92

(57) ABSTRACT

The present invention provides inhibitory molecules for inhibiting or at least partially preventing the binding of influenza virus hemagglutinin (HA) with Lymphocyte activation gene 3 (LAG-3) glycoprotein, for the treatment of influenza virus in a subject. The inhibition of this interaction may be achieved by binding to HA and/or by binding to LAG-3, e.g., by an antibody that binds specifically to the domain in LAG-3 that interacts with HA. The present invention also provides an antibody, or an antibody fragment thereof comprising at least the antigen binding portion, which specifically binds to an N-glycosylation site within human LAG-3, and use of the antibody or fragment thereof in treatment of a disease selected from cancer, autoimmune disease, inflammatory disease and a CNS disease.

### FIELD OF THE INVENTION

The present invention is in the fields of immunology and virology, and specifically, relates to inhibitors of T cell suppression pathways.

### BACKGROUND OF THE INVENTION

Influenza virus (also known as “flu”) is one of the few common infectious diseases which are poorly controlled by modern medicine. Influenza virus remains a global health concern, with millions of hospitalization cases and thousands of deaths reported annually. It is currently estimated that seasonal influenza may be involved in up to 650,000 deaths each year due to respiratory diseases.

Influenza is an enveloped segmented negative-sense RNA virus. As with all enveloped viruses, influenza virus requires the fusion of the viral and cellular membranes for entry into the cell, which is mediated by viral fusion proteins that protrude from the viral surface. For influenza virus, hemagglutinin (HA), a homotrimeric glycoprotein, is the viral fusion protein that mediates infection of epithelial cells in the respiratory system. The viral HA has two main functions that facilitate viral entry: 1) binding sialic acids on the surface of the target cells of the host, allowing the virus to attach to the cells and be internalized in endocytic vesicle, and 2) catalyzing the fusion of the viral membrane with the cell endosomal membrane following acid pH triggering (Samji T. (2009) Yale Jrnl Bio. and Med., Vol. 82). In the host infected cells, new viral HA glycoprotein are synthesized and are then transported to the plasma membrane for subsequent incorporation into nascent virions. Due to its critical role in infection, the HA is a main target of current influenza virus vaccines, and most of the current vaccines are designed to elicit neutralizing antibodies against this protein (Laursen and Wilson, (2013) Antiviral Research, Vol. 98; Carrat and Flahault (2007), Vaccine, Vol. 25; Nachbagauer and Krammer (2017) Clin. Microb, and Infect., Vol. 23).

The surface presentation of HA in infected cells can also initiate innate antiviral immune responses. For example, nature killer (NK) cells can recognize influenza virus-infected cells through the direct interaction of the NK activating receptors NKp44 and NKp46 with the viral HA, leading to NK-mediated elimination of the infected cells (Mandelboim et al. (2001), Nature. 409 (6823)). The HA glycoprotein is also recognized by non-neutralizing antibodies, which facilitate phagocytosis of the infected cells by macrophages and induce antibody-dependent cellular cytotoxicity (ADCC) by NK cells that eliminate the influenza virus-infected cells (Hullsiek et al. (2022), Front Immunol. Vol. 13). Thus, HA is a key glycoprotein in the virus life cycle, as well as in initiating an anti-viral immune response.

Following influenza virus infection, a vast immune response is elicited to limit the viral spread in the respiratory system (Chen et al. (2018), Front. in Immun., Vol. 9). However, influenza virus has developed several evasion mechanisms to counterattack and to escape these immune responses, allowing it to proliferate in the host (Quiñones-Parra et al. (2014), Front. in Microb., Vol 5). These diverse immune-evasion mechanisms, together with the high mutation rate of influenza virus (at least partially due to genetic reassortment), cause a situation in which the host immune responses and the currently available drugs often fail to control influenza virus infections, leading to regularly occurring epidemics.

The search for an effective therapeutic agent that will lead to sustained suppression of this virus is ongoing (Nachbagauer and Krammer (2017) Clin. Microb, and Infect., Vol. 23; Quiñones-Parra et al. (2014), Front. in Microb., Vol 5). Significant research and development of different types of anti-influenza vaccines has been going on for at least 50 years. Currently preferred quadrivalent vaccines, containing killed strains of types A and B virus which are in circulation at present, are administered seasonally to several billion people world-wide, but have only a 50 to 60% success rate in preventing infection (Nachbagauer and Krammer (2017) Clin. Microb. and Infect., Vol. 23). Standard influenza vaccines require annual redesigning, to counter new variants of the virus. In addition, the immunity which is provided by the vaccines is short-lived. Therefore, vaccines may not prevent or limit a pandemic if the circulating strains continue to drift significantly, or another subtype emerges. Instead, antiviral agents will be critical for initial control and protection against an emerging pandemic.

Much research has been directed towards developing anti-influenza neutralizing antibodies. Okuno et al. immunized mice with influenza A/Okuda/57 (H2N2) and isolated an antibody designated C179, which bound to a conserved conformational epitope in HA2 and neutralized the Group 1 subtype influenza A viruses in vitro and in vivo (Okuno et al. (1993) J. Virol. 67 (5): 2552-2558). Throsby et al. identified 13 monoclonal antibodies from human B cells that had broad activity against Group 1 subtypes (Throsby et al. (2008), PLOS one 3 (2): e3942). Sui et al. identified a human monoclonal antibody (F10), which bound H5 and other Group 1 viruses (Sui, et al. (2009), Nat. Struct. Mol. Biol. 16 (3): 265-273). After decades of research in this area, however, only relatively few antibodies are currently in clinical trials to assess their ability to neutralize different subtypes of influenza virus, and there are still no marketed antibodies that broadly neutralize or inhibit influenza A virus infection or attenuate the disease caused by various subtypes of this virus.

Other approaches to treatment of influenza virus, which do not involve anti-influenza antibodies, are directed towards blocking pathways which are involved in the infection and propagation of the virus, such as neuraminidase inhibitors (e.g., oseltamivir, zanamivir and the like), M2 ion-channel inhibitors (e.g., amantadine and rimantadine). However, several issues including adverse side effects, strain specific limitations, and swift emergence of resistant viral mutations, render these solutions incomplete and highlight the need for novel approaches (Jefferson et al., Cochrane Database Syst Rev 2014; 4; Samson, et al. Antiviral Res. 2013, 98 (2): 174-85; and Bright et al., Lancet. 2005. 366, 1175-1181)

Analysis of individuals prior to and after influenza virus infection have shown that clinical symptoms of the disease are inversely correlated with the frequency of influenza-specific T cells, defined as cells bearing a clonotypic TCR that recognizes conserved influenza viral epitopes. T cell-mediated cytotoxic activity plays a role in controlling infection, as cytotoxic CD8+ T-cell-deficient mice experience higher influenza-induced mortality, while the transfer of influenza-specific CD8+ T cells into naive mice was shown to protect mice from infection (Sant et al. (2018), Immun. Rev., Vol. 284). This protection is dependent on the ability of cytotoxic CD8+ T cell, by use of a T-cell receptor (TCR), to recognize a viral peptide presented in the context of Major Histocompatibility Complex (MHC) on the surface of the infected cells (Subbramanian et al. (2004), Nat Biotechnol., 22 (11)). A similar TCR-MHC interaction also initiates the activity of influenza-specific CD4 T cells, which promotes the production of neutralizing antibodies during infection, and is crucial for the elicitation of such antibodies following influenza virus vaccination (Sant et al. (2018), Immun. Rev., Vol. 284).

Lymphocyte activation gene 3 (LAG-3) is a single-pass transmembrane glycoprotein also known as CD223, expressed on a variety of immune cells. Lag-3 has been shown to negatively regulate the immune response, mainly by inhibiting T-cell activity and proliferation, and by reducing granzyme/cytokine production by T cells (Graydon et al. (2021) Front. in Immun., Vol. 11; Andrews et al. (2017), Immun. Rev., Vol. 276; Solinas et al. (2019), Cancers (Basel)., 11 (8)). The immunosuppressive role of LAG-3 has raised increasing interest in immunotherapies that target it, which may provide improved immune responses to cancer cells and to viral infections. However, at least partially due to the poor understanding of the ligands with which LAG-3 interacts, therapeutic agents against LAG-3 have not yet achieved significant clinical success. It is well established that the major histocompatibility complex class II (MHC-II) is one of the ligands that are recognized by LAG-3. Multiple other potential LAG-3 ligands have also been suggested, including C-type lectins (LSECtin/CLEC4G), Galectin3 (Gal-3), Fibrinogen-like protein 1 (FGL1), and α-synuclein (α-syn) (Graydon et al. (2021) Front. in Immun., Vol. 11; Andrews et al. (2017), Immun. Rev., Vol. 276; Solinas et al. (2019), Cancers (Basel)., 11 (8)). LAG-3 is not only expressed on the effector T cells but also on regulatory T cells and its antagonism can promote the activation of effector T cells but can also block the suppressive function of regulatory T cells. Therefore, LAG-3 represents a promising target for cancer immunotherapy and preclinical evidence suggests that anti-LAG-3 antibodies can promote an anti-tumor response.

Two of the LAG-3 ligands are lectins (proteins that bind to carbohydrates): C-type lectins and Galectin3 (Burnell, S. E. A. et al. Immunotherapy Advances 2, (2022), Kouo, T. et al. Cancer Immunol Res 3, (2015)). These ligands are expressed on tumor cells and in the central nervous system (CNS), thus, antibodies that bind LAG-3 may interfere with access of LAG-3 to the tumor and CNS ligands and may be used to treat tumors, autoimmune disorders, and central nervous system (CNS) diseases.

U.S. Pat. No. 11,414,485 (corresponding to WO2018/152687) teaches antibodies that bind Lymphocyte Activation Gene-3 (LAG-3). Also provided are methods of stimulating an immune response, inhibiting growth of tumor cells, and treating an autoimmune, inflammatory, or viral disease.

Despite the seeming suitability of LAG-3 to participate in immune interventions, the open questions related to the function of LAG-3 as a suppressive receptor limit the therapeutic potential of LAG-3 inhibitors.

There is an unmet need for an effective treatment for influenza virus infections, in particular a treatment which is effective for a variety of influenza strains, including newly emerging influenza strains. There is also an unmet need for developing novel antibodies that inhibit glycan-mediated LAG-3 interaction with relevant antigens, in a manner that stimulates an immune response that inhibits the growth of various cancers and tumor cells, as well as being useful in the treatment of autoimmune, inflammatory, or CNS diseases.

### SUMMARY OF THE INVENTION

The present invention provides inhibitory molecules for inhibiting or at least partially preventing glycan-dependent binding of influenza virus hemagglutinin (HA) with Lymphocyte activation gene 3 (LAG-3) glycoprotein. Molecules that inhibit this interaction are provided and include a mammalian LAG-3 fragment and conjugates and/or fusion proteins thereof, and antibodies specific to LAG-3, which target glycosylation binding sites on LAG-3. These inhibitory molecules may be used for the treatment of influenza virus in a subject. Furthermore, antibodies specific to human LAG-3 provided herein inhibit human LAG-3 glycan-dependent binding to human ligands and are useful in treatment of diseases and disorders that involves expression of human LAG-3, including cancers, autoimmune disorders, and CNS diseases.

The present invention is based in part on the unexpected discovery that the influenza virus HA glycoprotein directly interacts with a specific domain of the immune cells' LAG-3 glycoprotein, that the interaction between HA and LAG-3 has a hampering effect at least on the antiviral activity of CD8+ T-cells, and that blockage or inhibition of this interaction leads to an improved antiviral T cell response.

The present invention provides according to an aspect, a molecule that specifically inhibits the glycan-dependent binding of influenza hemagglutinin (HA) to mammalian Lymphocyte activation gene 3 (LAG-3). According to some embodiments, the molecule inhibits the binding of HA to LAG-3 by binding to influenza HA. According to some further embodiments, the molecule inhibits the binding of HA to LAG-3 by binding to LAG-3.

According to some embodiments, the molecule is a polypeptide.

According to some embodiments, the molecule comprises a mammalian LAG-3 fragment, conjugate, fusion protein, or combinations thereof. According to some embodiments, the LAG-3 fusion protein comprises a LAG-3 fragment or conjugate, fused to at least one carrier polypeptide. According to some embodiments, the carrier polypeptide is selected from human immunoglobulin and albumin, or fragments thereof. According to some embodiments, the human immunoglobulin is IgG1 or a fragment thereof. According to some further embodiments, the LAG-3 fragment, conjugate and/or fusion protein is fused to the polypeptide via a linker. According to some embodiments, the linker comprises 1-50 amino acid residues.

According to some particular embodiments, the LAG-3 fragment, conjugate and/or fusion protein comprises a fragment of LAG-3 fused to IgG1 Fc constant region. According to some embodiments, the fragment of LAG-3 comprises a sequence set forth in SEQ ID NO. 2. According to some embodiments, the fusion protein comprises the IgG1 Fc constant region comprising a sequence set forth in SEQ ID NO. 3. According to some embodiments, the fragment of LAG-3 fused to IgG1 Fc constant region comprises a sequence set forth in SEQ ID NO. 4.

According to some further embodiments, the LAG-3 fragment, conjugate or fusion protein comprises at least one N-glycosylation site. According to some further embodiments, the LAG-3 N-glycosylation site is located on an amino acid selected from the group consisting of: amino acid 188, amino acid 250, amino acid 256, and amino acid 343, of SEQ ID NO. 1.

According to some embodiments, the molecule that inhibits the binding of HA to LAG-3 by binding to LAG-3 is an antibody specific to LAG-3, or a fragment or conjugate thereof comprising at least the binding site. According to some further embodiments, the monoclonal antibody is configured to bind at least one LAG-3 N-glycosylation site. According to some particular embodiments, the at least one LAG-3 N-glycosylation site is located within residues 169-351 of SEQ ID NO. 1. According to some particular embodiments, the at least one LAG-3 N-glycosylation site is located within residues 169-260 of SEQ ID NO. 1. According to some particular embodiments, the at least one LAG-3 N-glycosylation site is located within residues 263-351 of SEQ ID NO. 1. According to some particular embodiments, the at least one LAG-3 N-glycosylation site is located on an amino acid selected from the list consisting of: amino acid 188, amino acid 250, amino acid 256, and amino acid 343, of SEQ ID NO. 1.

According to some embodiments, the antibody or antibody fragment binds to an epitope comprising at least one N-glycosylation site within the D2 domain of human LAG-3 (SEQ ID NO. 6).

According to other embodiments, the antibody or antibody fragment binds to an epitope comprising an N-glycosylation site at position 343 of SEQ ID NO. 1.

According to some embodiments, the antibody is a monoclonal antibody (mAb) or a fragment or conjugate thereof comprising at least the binding site.

According to some embodiments the antibody is a chimeric antibody.

According to some embodiments, the chimeric antibody comprises human constant regions. According to some specific embodiments, the chimeric antibody comprises human kappa light chain and human IgG1 heavy chain.

According to some embodiments, the antibody fragment is selected from Fab and scFv.

Humanized antibodies comprising the CDR sequences of the anti-human LAG-3 antibodies disclosed herein are also within the scope of the present invention.

The present invention provides, according to another aspect, an antibody specific to the D2 domain (SEQ ID NO. 6) of LAG-3, or a fragment or conjugate thereof comprising at least the binding site.

According to some embodiments, the antibody or antibody fragment comprises a set of six complementarity determining region (CDR) sequences selected from the group consisting of:
i. SEQ ID No. 35, YTS, SEQ ID No. 36, SEQ ID No. 37, SEQ ID No. 38, and SEQ ID No. 39;
ii. SEQ ID No. 35, YTS, SEQ ID No. 40, SEQ ID No. 41, SEQ ID No. 42, and SEQ ID No. 43;
iii. 35, YTS, 44, 41, 42, and SEQ ID No. 43;
iv. 45, AAT, 46, 41, 42, and SEQ ID No. 43;
v. 47, STS, 48, 41, 42, and SEQ ID No. 43;
vi. 47, STS, 48, 41, 42, and SEQ ID No. 49;
vii. 50, AAS, 51, 41, 42, and SEQ ID No. 43;
viii. 50, AAS, 51, 37, 38, and SEQ ID No. 52;
ix. 53, SAS, 54, 41, 42, and SEQ ID No. 43;
x. 53, SAS, 54, 76, 55, and SEQ ID No. 56;
xi. 57, WTS, 58, 41, 42, and SEQ ID No. 43;
xii. 59, KVS, 60, 41, 42, and SEQ ID No. 43;
xiii. 35, YTS, 36, 41, 42, and SEQ ID No. 43;
xiv. 45, AAT, 61, 41, 42, and SEQ ID No. 43;
xv. 62, LVS, 63, 41, 42, and SEQ ID No. 43;
xvi. 62, LVS, 63, 64, 65, and SEQ ID No. 66;
xvii. 67, RCT, 68, 41, 42, and SEQ ID No. 43;
xviii. 45, AAT, 69, 41, 42, and SEQ ID No. 43;
xix. 70, AAS, 71, 41, 42, and SEQ ID No. 43;
xx. 70, AAS, 71, 41, 72, and SEQ ID No. 73;
xxi. 74, DTS, 75, 41, 42, and SEQ ID No. 43;
xxii. 74, DTS, 75, 76, 77, and SEQ ID No. 78;
xxiii. 59, KVS, 79, 41, 42, and SEQ ID No. 43;
xxiv. 62, LVS, 80, 41, 42, and SEQ ID No. 43.

Each option represents a separate embodiment of the present invention.

According to some embodiments, the antibody or antibody fragment comprises a combination of a light chain variable region (LC-VR) and a heavy chain variable region (HC-VR), wherein the combination is selected from:
i. SEQ ID Nos. 9 and 10;
ii. SEQ ID Nos. 11 and 12;
iii. SEQ ID Nos. 13 and 12;
iv. SEQ ID Nos. 14 and 12;
v. SEQ ID Nos. 15 and 12;
vi. SEQ ID Nos. 15 and 16;
vii. SEQ ID Nos. 17 and 12;
viii. SEQ ID Nos. 17 and 18;
ix. SEQ ID Nos. 19 and 12;
x. SEQ ID Nos. 19 and 20;
xi. SEQ ID Nos. 21 and 12;
xii. SEQ ID Nos. 22 and 12;
xiii. SEQ ID Nos. 9 and 12;
xiv. SEQ ID Nos. 23 and 12;
xv. SEQ ID Nos. 24 and 12;
xvi. SEQ ID Nos. 24 and 25;
xvii. SEQ ID Nos. 26 and 12;
xviii. SEQ ID Nos. 26 and 27;
xix. SEQ ID Nos. 28 and 12;
xx. SEQ ID Nos. 29 and 12;
xxi. SEQ ID Nos. 29 and 30;
xxii. SEQ ID Nos. 31 and 12;
xxiii. SEQ ID Nos. 31 and 32;
xxiv. SEQ ID Nos. 33 and 12;
xxv. SEQ ID Nos. 34 and 12.
or an analog of any of (i)-(xxv) having at least 90% sequence identity with the LC-VR and HC-VR sequences; Each option represents a separate embodiment of the present invention.

According to some embodiments, the antibody or antibody fragment binds to an epitope within the D2 domain and comprises a combination of a LC-VR and a HC-VR, wherein the combination is selected from:
i. SEQ ID Nos. 9 and 10;
ii. SEQ ID Nos. 11 and 12;
iii. SEQ ID Nos. 13 and 12;
iv. SEQ ID Nos. 14 and 12;
v. SEQ ID Nos. 15 and 12;
vi. SEQ ID Nos. 15 and 16;
vii. SEQ ID Nos. 17 and 12;
viii. SEQ ID Nos. 17 and 18;
ix. SEQ ID Nos. 19 and 12;
x. SEQ ID Nos. 19 and 20.

Each option represents a separate embodiment of the present invention.

According to some embodiments, the antibody or antibody fragment comprises a HC-VR comprising SEQ ID No. 12 and a LC-VR comprising a sequence selected from SEQ ID Nos. 11, 13, 14, 15, 17, 19, 21, 22, 9, 23, 24, 26, 28, 29, 33 and 34. Each option represents a separate embodiment of the present invention.

The present invention further provides a polynucleotide sequence encoding a molecule that specifically inhibits the binding of influenza hemagglutinin (HA) to mammalian Lymphocyte activation gene 3 (LAG-3).

According to some embodiments, the polynucleotide encodes a molecule that inhibits the binding of HA to LAG-3 by binding to influenza HA.

According to some further embodiments, the polynucleotide encodes at least one chain of an antibody that inhibits the binding of HA to LAG-3 by binding to LAG-3.

According to some embodiments, the polynucleotide encodes a LC-VR sequence of an antibody or antibody fragment, the polynucleotide is selected from the group consisting of SEQ ID Nos. 81, 83, 85, 86, 87, 89, 91, 93, 94, 95, 96, 98, 100, 101, 103, 105 and 106 or an analog or derivative thereof having at least 90% sequence identity with the variable region encoding sequences. Each possibility represents a separate embodiment of the present invention.

According to some embodiments, the polynucleotide encodes a HC-VR sequence of an antibody or antibody fragment, the polynucleotide is selected from the group consisting of SEQ ID Nos. 82, 84, 88, 90, 92, 97, 99, 102, and 104, or an analog or derivative thereof having at least 90% sequence identity with the variable region encoding sequences. Each possibility represents a separate embodiment of the present invention.

According to some embodiments, pairs of polynucleotides encoding amino acid sequences comprising heavy and light chain variable regions described above are provided.

According to some embodiments, a plasmid or vector comprising one or more of the polynucleotide sequences described above are provided, as well as a cell or population of cells comprising one or more of the polynucleotide sequence or one or more plasmids or vectors comprising them. According to some embodiments, the plasmid or vector comprises two polynucleotides, one polynucleotide encoding an amino acid sequence comprising a heavy chain variable region and one polynucleotide encoding an amino acid sequence comprising a light chain variable region.

According to some embodiments, the molecule is for use in inhibiting the propagation or activity of influenza virus.

According to some embodiments, the molecule is for use in the treatment of influenza virus infection.

The present invention provides, according to yet another aspect, a pharmaceutical composition comprising at least one molecule that specifically inhibits the binding of influenza hemagglutinin (HA) to mammalian Lymphocyte activation gene 3 (LAG-3), and a pharmaceutically acceptable excipient, diluent, salt and/or buffer.

According to some embodiments, the pharmaceutical composition is for use in prevention, amelioration, or treatment of influenza virus infection. According to some embodiments, the influenza virus is selected from influenza A, and influenza B. According to some particular embodiments, the Influenza virus comprises at least one of influenza A H1N1, influenza A H5N1, and influenza A H3N2.

According to some further embodiments, the pharmaceutical composition is for use as part of a treatment regimen in conjunction with at least one anti-influenza composition or therapy.

The present invention provides, according to yet another aspect, a method of treating or preventing a disease or disorder caused by an influenza virus, the method comprising administering to a subject at risk of suffering from said disease or disorder, a therapeutically effective amount of a molecule that specifically inhibits the binding of influenza hemagglutinin (HA) to mammalian Lymphocyte activation gene 3 (LAG-3).

The present invention provides, according to yet another aspect, a method of treating or preventing a disease or disorder caused by an influenza virus, comprising contacting the influenza virus with a molecule that specifically inhibits the binding of influenza hemagglutinin (HA) to mammalian Lymphocyte activation gene 3 (LAG-3). According to some embodiments, the molecule comprises a mammalian LAG-3 fragment, conjugate and/or fusion protein.

The present invention provides, according to yet another aspect, a method of treating or preventing a disease or disorder caused by an influenza virus, comprising contacting CD8+ T cells with a molecule that specifically inhibits the binding of influenza hemagglutinin (HA) to mammalian Lymphocyte activation gene 3 (LAG-3). According to some embodiments, the molecule comprises a monoclonal antibody (mAb) specific to LAG-3, or a fragment thereof comprising at least the binding site.

The present invention also provides, according to another aspect, antibodies capable of binding to human LAG-3 protein, and fragments thereof comprising at least the antigen binding portion, as well as conjugates and pharmaceutical compositions comprising them and their uses in therapeutic diagnostic and analytical settings. Some of the anti-LAG-3 antibodies disclosed herein exhibited activities not shown with known anti-LAG-3 antibodies, in particular, binding to N-glycosylation sites in specific domains of the protein and inhibition of glycan-mediated binding to viral antigens such as the influenza virus HA.

According to another aspect, the present invention provides an antibody, or an antibody fragment thereof comprising at least the antigen binding portion, which specifically binds to an N-glycosylation site of human LAG-3, said antibody or fragment thereof comprising a set of six CDR sequences wherein the set is selected from the group consisting of:
i. three CDRs of a heavy-chain (HC) variable region comprising SEQ ID NO. 9 and three CDRs of a light-chain (LC) variable comprising SEQ ID NO. 10;
ii. three CDRs of a heavy-chain variable region comprising SEQ ID NO. 11 and three CDRs of a light-chain variable region comprising SEQ ID NO. 12;
iii. three CDRs of a heavy-chain variable region comprising SEQ ID NO. 13 and three CDRs of a light-chain variable region comprising SEQ ID NO. 12;
iv. three CDRs of a heavy-chain variable region comprising SEQ ID NO. 14 and three CDRs of a light-chain variable region comprising SEQ ID NO. 12;
v. three CDRs of a heavy-chain variable region comprising SEQ ID NO. 15 and three CDRs of a light-chain variable region comprising SEQ ID NO. 12;
vi. three CDRs of a heavy-chain variable region comprising SEQ ID NO. 15 and three CDRs of a light-chain variable region comprising SEQ ID NO. 16;
vii. three CDRs of a heavy-chain variable region comprising SEQ ID NO. 17 and three CDRs of a light-chain variable region comprising SEQ ID NO. 12;
viii. three CDRs of a heavy-chain variable region comprising SEQ ID NO. 17 and three CDRs of a light-chain variable region comprising SEQ ID NO. 18;
ix. three CDRs of a heavy-chain variable region comprising SEQ ID NO. 19 and three CDRs of a light-chain variable region comprising SEQ ID NO. 12;
x. three CDRs of a heavy-chain variable region comprising SEQ ID NO. 19 and three CDRs of a light-chain variable region comprising SEQ ID NO. 20;
xi. three CDRs of a heavy-chain variable region comprising SEQ ID NO. 21 and three CDRs of a light-chain variable region comprising SEQ ID NO. 12;
xii. three CDRs of a heavy-chain variable region comprising SEQ ID NO. 22 and three CDRs of a light-chain variable region comprising SEQ ID NO. 12;
xiii. three CDRs of a heavy-chain variable region comprising SEQ ID NO. 9 and three CDRs of a light-chain variable region comprising SEQ ID NO. 12;
xiv. three CDRs of a heavy-chain variable region comprising SEQ ID NO. 23 and three CDRs of a light-chain variable region comprising SEQ ID NO. 12;
xv. three CDRs of a heavy-chain variable region comprising SEQ ID NO. 24 and three CDRs of a light-chain variable region comprising SEQ ID NO. 12;
xvi. three CDRs of a heavy-chain variable region comprising SEQ ID NO. 24 and three CDRs of a light-chain variable region comprising SEQ ID NO. 25;
xvii. three CDRs of a heavy-chain variable region comprising SEQ ID NO. 26 and three CDRs of a light-chain variable region comprising SEQ ID NO. 12;
xviii. three CDRs of a heavy-chain variable region comprising SEQ ID NO. 26 and three CDRs of a light-chain variable region comprising SEQ ID NO. 27;
xix. three CDRs of a heavy-chain variable region comprising SEQ ID NO. 28 and three CDRs of a light-chain variable region comprising SEQ ID NO. 12;
xx. three CDRs of a heavy-chain variable region comprising SEQ ID NO. 29 and three CDRs of a light-chain variable region comprising SEQ ID NO. 12;
xxi. three CDRs of a heavy-chain variable region comprising SEQ ID NO. 29 and three CDRs of a light-chain variable region comprising SEQ ID NO. 30;
xxii. three CDRs of a heavy-chain variable region comprising SEQ ID NO. 31 and three CDRs of a light-chain variable region comprising SEQ ID NO. 12;
xxiii. three CDRs of a heavy-chain variable region comprising SEQ ID NO. 31 and three CDRs of a light-chain variable region comprising SEQ ID NO. 32;
xxiv. three CDRs of a heavy-chain variable region comprising SEQ ID NO. 33 and three CDRs of a light-chain variable region comprising SEQ ID NO. 12; and
xxv. three CDRs of a heavy-chain variable region comprising SEQ ID NO. 34 and three CDRs of a light-chain variable region comprising SEQ ID NO. 12.
   Each option represents a separate embodiment of the present invention.

There are several methods known in the art for determining the CDR sequences of a given antibody molecule, but there is no standard unequivocal method. Determination of CDR sequences from antibody heavy and light chain variable regions can be made according to any method known in the art, including, but not limited to, the methods known as KABAT, Chothia, and IMGT. A selected set of CDRs may include sequences identified by more than one method, namely, some CDR sequences may be determined using KABAT and some using IMGT, for example. According to some embodiments, the CDR sequences of the mAb variable regions are determined using the KABAT and/or Chothia methods. The use of other methods of determination of CDR sequences, including in-silico programs and screening of public and inhouse databases, is also optional within the scope of the present invention.

According to some embodiments, the antibody inhibits binding of human LAG-3 to at least one molecule selected from: influenza hemagglutinin, Galectin-3 (LGALS3), C-type lectin domain family 4 member G (LSECtin) protein, MHC class II molecules, Fibrinogen-like protein 1 (FGL1), and Alpha-synuclein pff (α-Syn PFF).

According to some embodiments, the antibody or antibody fragment binds to an N-glycosylation site at position 343 of SEQ ID NO. 1.

According to some embodiments, the antibody or antibody fragment comprises a combination of a light chain variable region (LC-VR) and a heavy chain variable region (HC-VR), wherein the combination is selected from:
i. SEQ ID Nos. 9 and 10;
ii. SEQ ID Nos. 11 and 12;
iii. SEQ ID Nos. 13 and 12;
iv. SEQ ID Nos. 14 and 12;
v. SEQ ID Nos. 15 and 12;
vi. SEQ ID Nos. 15 and 16;
vii. SEQ ID Nos. 17 and 12;
viii. SEQ ID Nos. 17 and 18;
ix. SEQ ID Nos. 19 and 12;
x. SEQ ID Nos. 19 and 20;
xi. SEQ ID Nos. 21 and 12;
xii. SEQ ID Nos. 22 and 12;
xiii. SEQ ID Nos. 9 and 12;
xiv. SEQ ID Nos. 23 and 12;
xv. SEQ ID Nos. 24 and 12;
xvi. SEQ ID Nos. 24 and 25;
xvii. SEQ ID Nos. 26 and 12;
xviii. SEQ ID Nos. 26 and 27;
xix. SEQ ID Nos. 28 and 12;
xx. SEQ ID Nos. 29 and 12;
xxi. SEQ ID Nos. 29 and 30;
xxii. SEQ ID Nos. 31 and 12;
xxiii. SEQ ID Nos. 31 and 32;
xxiv. SEQ ID Nos. 33 and 12;
xxv. SEQ ID Nos. 34 and 12.
or an analog of any of (i)-(xxv) having at least 90% sequence identity with the LC-VR and HC-VR sequences. Each option represents a separate embodiment of the present invention.

According to some embodiments, the antibody or antibody fragment comprises a combination of a LC-VR and a HC-VR, wherein the combination is selected from:
i. SEQ ID Nos. 9 and 10;
ii. SEQ ID Nos. 11 and 12;
iii. SEQ ID Nos. 13 and 12;
iv. SEQ ID Nos. 14 and 12;
v. SEQ ID Nos. 15 and 12;
vi. SEQ ID Nos. 15 and 16;
vii. SEQ ID Nos. 17 and 12;
viii. SEQ ID Nos. 17 and 18;
ix. SEQ ID Nos. 19 and 12;
x. SEQ ID Nos. 19 and 20.

Each option represents a separate embodiment of the present invention.

According to some embodiments, the antibody or antibody fragment comprises a HC-VR comprising SEQ ID No. 12 and a LC-VR comprising a sequence selected from SEQ ID Nos. 11, 13, 14, 15, 17, 19, 21, 22, 9, 23, 24, 26, 28, 29, 33 and 34. Each option represents a separate embodiment of the present invention.

According to some embodiments, the antibody or antibody fragment comprises a HC-VR of SEQ ID No. 12 and a LC-CR of a sequence selected from SEQ ID Nos. 11, 13, 14, 15, 17, 19.

Each option represents a separate embodiment of the present invention.

According to some embodiments, the antibody or antibody fragment comprises a set of 6 CDR sequences (LC CDR1, LC-CDR2, LC-CDR3, HC-CDR-1, HC-CDR2, HC-CDR-3), the set is selected from:
i. SEQ ID No. 35, YTS, SEQ ID No. 36, SEQ ID No. 37, SEQ ID No. 38, and SEQ ID No. 39;
ii. SEQ ID No. 35, YTS, SEQ ID No. 40, SEQ ID No. 41, SEQ ID No. 42, and SEQ ID No. 43;
iii. 35, YTS, 44, 41, 42, and SEQ ID No. 43;
iv. 45, AAT, 46, 41, 42, and SEQ ID No. 43;
v. 47, STS, 48, 41, 42, and SEQ ID No. 43;
vi. 47, STS, 48, 41, 42, and SEQ ID No. 49;
vii. 50, AAS, 51, 41, 42, and SEQ ID No. 43;
viii. 50, AAS, 51, 37, 38, and SEQ ID No. 52;
ix. 53, SAS, 54, 41, 42, and SEQ ID No. 43;
x. 53, SAS, 54, 76, 55, and SEQ ID No. 56;
xi. 57, WTS, 58, 41, 42, and SEQ ID No. 43;
xii. 59, KVS, 60, 41, 42, and SEQ ID No. 43;
xiii. 35, YTS, 36, 41, 42, and SEQ ID No. 43;
xiv. 45, AAT, 61, 41, 42, and SEQ ID No. 43;
xv. 62, LVS, 63, 41, 42, and SEQ ID No. 43;
xvi. 62, LVS, 63, 64, 65, and SEQ ID No. 66;
xvii. 67, RCT, 68, 41, 42, and SEQ ID No. 43;
xviii. 45, AAT, 69, 41, 42, and SEQ ID No. 43;
xix. 70, AAS, 71, 41, 42, and SEQ ID No. 43;
xx. 70, AAS, 71, 41, 72, and SEQ ID No. 73;
xxi. 74, DTS, 75, 41, 42, and SEQ ID No. 43;
xxii. 74, DTS, 75, 76, 77, and SEQ ID No. 78;
xxiii. 59, KVS, 79, 41, 42, and SEQ ID No. 43;
xxiv. 62, LVS, 80, 41, 42, and SEQ ID No. 43.

Each option represents a separate embodiment of the present invention.

According to some embodiments, the antibody or antibody fragment that binds to an N-glycosylation site within the D2 domain (SEQ ID NO 6), of human LAG-3 comprises a set of six CDR sequences, wherein the set is selected from:
i. SEQ ID No. 35, YTS, SEQ ID No. 36, SEQ ID No. 37, SEQ ID No. 38, and SEQ ID No. 39;
ii. SEQ ID No. 35, YTS, SEQ ID No. 40, SEQ ID No. 41, SEQ ID No. 42, and SEQ ID No. 43;
iii. 35, YTS, 44, 41, 42, and SEQ ID No. 43;
iv. 45, AAT, 46, 41, 42, and SEQ ID No. 43;
v. 47, STS, 48, 41, 42, and SEQ ID No. 43;
vi. 47, STS, 48, 41, 42, and SEQ ID No. 49;
vii. 50, AAS, 51, 41, 42, and SEQ ID No. 43;
viii. 50, AAS, 51, 37, 38, and SEQ ID No. 52;
ix. 53, SAS, 54, 41, 42, and SEQ ID No. 43;
x. 53, SAS, 54, 76, 55, and SEQ ID No. 56.

Each option represents a separate embodiment of the present invention.

According to some embodiments, the antibody comprises the six CDR sequences of a monoclonal antibody selected from the group consisting of: 23DKTYB4; 23DKTYB10; 23DKTYB11; 23DKTYB13; 23DKTYB14; 23DKTYB15; 23DKTYB18; 23DKTYB19; 23DKTYB20; 23DKTYB21; 23DKTYB1; 23DKTYB2; 23DKTYB3; 23DKTYB5; 23DKTYB6; 23DKTYB7; 23DKTYB8; 23DKTYB9; 23DKTYB12; 23DKTYB16; 23DKTYB17; 23DKTYB22; 23DKTYB23; 23DKTYB24; 23DKTYB25. Each option represents a separate embodiment of the present invention.

According to some embodiments, the antibody or the antibody fragment comprises a CDR set selected from the group consisting of:
i. SEQ ID No. 35, YTS, SEQ ID No. 36, SEQ ID No. 37, SEQ ID No. 38, and SEQ ID No. 39;
ii. SEQ ID No. 35, YTS, SEQ ID No. 40, SEQ ID No. 41, SEQ ID No. 42, and SEQ ID No. 43;
iii. 35, YTS, 44, 41, 42, and SEQ ID No. 43; 45, AAT, 46, 41, 42, and SEQ ID No. 43; iv.
v. 47, STS, 48, 41, 42, and SEQ ID No. 43;
vi. 47, STS, 48, 41, 42, and SEQ ID No. 49;
vii. 50, AAS, 51, 41, 42, and SEQ ID No. 43;
viii. 50, AAS, 51, 37, 38, and SEQ ID No. 52;
ix. 53, SAS, 54, 41, 42, and SEQ ID No. 43;
x. 53, SAS, 54, 76, 55, and SEQ ID No. 56;
xi. 57, WTS, 58, 41, 42, and SEQ ID No. 43;
xii. 59, KVS, 60, 41, 42, and SEQ ID No. 43;
xiii. 35, YTS, 36, 41, 42, and SEQ ID No. 43;
xiv. 45, AAT, 61, 41, 42, and SEQ ID No. 43;
xv. 62, LVS, 63, 41, 42, and SEQ ID No. 43;
xvi. 62, LVS, 63, 64, 65, and SEQ ID No. 66;
xvii. 67, RCT, 68, 41, 42, and SEQ ID No. 43;
xviii. 45, AAT, 69, 41, 42, and SEQ ID No. 43;
xix. 70, AAS, 71, 41, 42, and SEQ ID No. 43;
xx. 70, AAS, 71, 41, 72, and SEQ ID No. 73;
xxi. 74, DTS, 75, 41, 42, and SEQ ID No. 43;
xxii. 74, DTS, 75, 76, 77, and SEQ ID No. 78;
xxiii. 59, KVS, 79, 41, 42, and SEQ ID No. 43;
xxiv. 62, LVS, 80, 41, 42, and SEQ ID No. 43.

or an analog thereof comprising no more than 5% amino acid substitutions, deletions and/or insertions in the hypervariable region (HVR) sequence that comprises the six CDRs.

According to some embodiments, the antibody or fragment thereof recognizes human LAG-3 with an affinity of at least 10⁻⁸M. According to other embodiments, the antibody or antibody fragment binds human LAG-3 with affinity of at least 109M, or even higher, to human LAG-3. According to some embodiments, the antibody or antibody fragment binds to human LAG-3 with affinity in the range of 10⁻⁹M to 10-12M. According to some embodiments, the antibody or antibody fragment binds to human LAG-3 with affinity in the range of 10⁻⁸M to 10-12M. Each possibility represents a separate embodiment of the invention.

Analogs, variants and derivatives of the antibody and the fragments described above, are also within the scope of the invention, e.g., derivatives, variants, and analogs of the antibodies described above.

According to some embodiments, the antibody or antibody fragment analog has at least 95% sequence identity with the hypervariable region of the reference antibody sequence.

According to certain embodiments, the analog or derivative of the isolated antibody or fragment thereof has at least 91, 92, 93, 94, 95, 96, 97, 98 or 99% sequence identity with a variable region of the reference antibody sequence. Each possibility represents a separate embodiment of the invention.

Analogs of antibodies and antibody fragments comprising a combination of variable regions described above, having at least 95% sequence similarity with said heavy or light chain variable regions are also included within the scope of the present invention.

According to some embodiments, the analog has at least 96, 97, 98 or 99% sequence similarity or identity with an antibody light or heavy chain variable regions described above. According to some embodiments, the analog comprises no more than one amino acid substitution, deletion or addition to one or more CDR sequences of a hypervariable region disclosed above. Each possibility represents a separate embodiment of the present invention. According to some embodiments, the amino acid substitution is a conservative substitution.

According to some embodiments, the antibody or antibody fragment comprises a hypervariable region (HVR) having light and heavy chain regions defined above, in which 1, 2, 3, 4, or 5 amino acids were substituted, deleted and/or added. Each possibility represents a separate embodiment of the invention.

According to some embodiments, the antibody or antibody fragment comprises a HVR having light and heavy chain regions defined above, in which one amino acid was substituted. According to specific embodiments, the antibody or antibody fragment comprises a CDR as defined above, in which one amino acid was substituted.

According to some embodiments, the antibody is an isolated monoclonal antibody (mAb).

According to some embodiments, the isotype of the antibody is mouse IgG1/Kappa.

According to specific embodiments, the antibody is a chimeric antibody.

According to yet other embodiments, the chimeric antibody comprises of human-derived constant region.

According to some embodiments, the chimeric mAb comprises a human constant region selected from the group consisting of: human IgG1, human IgG2, human IgG3 and human IgG4. Each possibility represents a separate embodiment of the present invention.

According to specific embodiments, the antibody is an antibody fragment. According to a specific embodiment, the antibody fragment is selected from the group consisting of: Fab, Fab′, F(ab′)₂, Fd, Fd′, Fv, dAb, isolated CDR region, single chain variable fragment (scFv), single chain antibody (scab), “diabodies”, and “linear antibodies”. Each possibility represents a separate embodiment of the present invention.

According to specific embodiments, the scFv comprises the heavy and light chains of an antibody described herein. According to some embodiments, the scFv molecule comprises the antigen binding site of the antibody expressed in one polypeptide chain. According to some embodiments, the invention provides scFv molecules comprising a heavy chain and a light chain variable regions of the anti-LAG-3 antibodies. According to certain embodiments, the scFv comprises a hinge region between the two variable regions.

According to some specific embodiments, the antibody fragment is a Fab or a scFv.

The present invention also provides humanized antibodies comprising a set of six CDRs of any of the antibodies described herein.

According to some embodiments, a conjugate comprising a mAb or a fragment thereof as described herein is provided.

A conjugate according to some embodiments of the present invention comprises an antibody or fragment thereof defined above, attached directly or through a spacer or a linker to a moiety including but not limited to, a radioactive moiety, a labeling tag and a cytotoxic moiety.

Polynucleotides encoding amino acid chains of antibodies and antibody fragment that bind human LAG-3, having the specific CDR sequences detailed above, as well as vectors and host cells carrying these polynucleotides, are provided according to another aspect of the present invention.

According to some embodiments, polynucleotides encoding the amino acid sequences of heavy chain variable regions and light chain variable regions described above, and heavy and light chain comprising them are provided.

According to some embodiments, the polynucleotide that encodes an antibody or antibody fragment comprising a LC-VR, comprises a sequence selected from the group consisting of SEQ ID Nos. 81, 83, 85, 86, 87, 89, 91, 93, 94, 95, 96, 98, 100, 101, 103, 105, and 106, or an analog or derivative thereof having at least 90% sequence identity with the variable region encoding sequences. Each possibility represents a separate embodiment of the present invention.

According to some embodiments, the polynucleotide that encodes an antibody or antibody fragment comprising a HC-VR, comprises a sequence selected from the group consisting of SEQ ID Nos. 82, 84, 88, 90, 92, 97, 99, 102, and 104, or an analog or derivative thereof having at least 90% sequence identity with the variable region encoding sequences. Each possibility represents a separate embodiment of the present invention.

According to some embodiments, the polynucleotides defined above encode a molecule selected from the group consisting of: an antibody, an antibody fragment comprising at least an antigen-binding portion, an antibody chain, and an antibody conjugate comprising said antibody or antibody fragment. Each possibility represents a separate embodiment of the present invention.

According to some embodiments, the polynucleotide encodes both the heavy chain and the light chain, or the heavy chain variable region and the light chain variable region of an antibody described above.

The present invention provides, according to some embodiments, a polypeptide comprising at least one sequence encoded by at least one polynucleotide sequence disclosed above.

In a further aspect, the present invention provides a nucleic acid construct comprising a polynucleotide encoding at least one antibody chain or fragment thereof as described herein. According to some embodiments the nucleic acid construct is a plasmid.

According to some embodiments, the vector or plasmid comprises a polynucleotide encoding a heavy chain or a heavy chain variable region and a polynucleotide encoding a light chain or a light chain variable region.

According to some embodiments, the vector or plasmid comprises a polynucleotide encoding both the heavy chain and the light chain or the heavy chain variable region and the light chain variable region of an antibody.

In still another aspect the present invention provides a cell capable of producing an antibody or an antibody fragment comprising the specific CDR sequences and/or specific heavy and light chain variable regions described herein.

According to some embodiments, a cell or a population of cells is provided comprising at least one polynucleotide, construct or vector disclosed above.

According to some embodiments, the cell producing a monoclonal antibody described above is a hybridoma cell.

The present invention provides, according to another aspect, a pharmaceutical composition comprising as an active ingredient, at least one antibody, antibody fragment or conjugate thereof, as described herein, and optionally at least one pharmaceutical acceptable excipient, diluent, salt, or carrier.

The present invention also provides, according to yet other aspect, a monoclonal antibody or a fragment or conjugate thereof, or a pharmaceutical composition comprising any of them, for use in inhibiting a glycan-dependent binding of human LAG-3 to a ligand. According to some embodiments, the ligand is expressed on tumor cells or in the central nervous system (CNS). According to some embodiments, the ligand is influenza HA. According to other embodiments, the LAG-3 ligand is a lectin. According to some specific embodiments, the lectin is a human C-type lectin (LSECtin/CLEC4G) or human Galectin3 (Gal-3).

According to some embodiments, the pharmaceutical composition is for use in preventing, attenuating or treating a disease or disorder associated with LAG-3 expression, overexpression or activity or with binding of LAG-3 to any of its ligands. According to some embodiments, the LAG-3 ligand is influenza HA. According to other embodiments, the LAG-3 ligand is a lectin. According to some specific embodiments, the lectin is a human C-type lectin (LSECtin/CLEC4G) or human Galectin3 (Gal-3). According to some further embodiments, the LAG-3 ligand is a human MHC class II molecule. According to yet further embodiments, the LAG-3 ligand is a human Fibrinogen-like protein 1 (FGL1). According to further embodiments, the LAG-3 ligand is a human Alpha-synuclein pff (α-Syn PFF).

According to some embodiments, the disease or disorder associated with human LAG-3 expression is selected from the group including but not limited to: influenza, cancer, autoimmune disease, infectious disease and neurogenerative disease.

According to some embodiments, the neurogenerative disease is a CNS disease selected from Parkinson's disease and Alzheimer's disease.

According to some embodiments, the disease is cancer or tumor.

According to some embodiments of the present invention, the pharmaceutical composition is for use in cancer immunotherapy.

According to some embodiments of the invention, the cancer is selected from the group consisting of a lung cancer, a breast cancer, a colorectal cancer, a melanoma, an ovarian cancer, a pancreatic cancer, a colon cancer, a cervical cancer, a kidney cancer, a thyroid cancer, a prostate cancer, a brain cancer, a renal cancer, a throat cancer, a laryngeal carcinoma, a bladder cancer, a hepatic cancer, a fibrosarcoma, an endometrial cells cancer, a glioblastoma, and a sarcoma. Each possibility represents a separate embodiment of the invention. According to some embodiments, the pharmaceutical composition is for use in treating a cancer or tumor selected from the group consisting of Melanoma, Hodgkin's lymphoma, Diffuse large B-cell lymphoma, Urothelial cancer, Colorectal cancer, Hepatocellular carcinoma, Non-small cell lung cancer, Small cell lung cancer, Renal cell carcinoma, Squamous cell carcinoma, Gastric cancer, Esophageal cancer, Cutaneous squamous cell carcinoma, Triple negative breast cancer, and Merkel cell carcinoma. Each possibility represents a separate embodiment of the invention.

According to some embodiments, the cancer is a solid cancer or comprises a solid tumor. According to some specific embodiments, the cancer is selected from the group consisting of breast cancer, colorectal cancer, lung cancer, bladder cancer, pancreatic cancer, ovarian cancer, kidney cancer, melanoma, prostate cancer, and brain cancer. Each possibility represents a separate embodiment of the invention.

According to some embodiments, the cancer or tumor is selected from the group consisting of renal cancer, ovarian cancer, colon cancer, prostate cancer, and breast cancer. According to some embodiments, the cancer or tumor is selected from the group consisting of renal cancer, colon cancer, prostate cancer and breast cancer. Each possibility represents a separate embodiment of the invention.

According to other embodiments, the cancer is hematologic cancer selected from a myeloma, a leukemia, and a lymphoma.

According to some embodiments, the cancer is a metastatic cancer.

According to some embodiments, the pharmaceutical composition is for use in inhibiting a metastatic cascade, namely preventing, reducing or inhibiting metastases formation, migration, adhesion, spread, and/or growth.

According to yet another aspect, the present invention provides a method of preventing, attenuating, delaying, or treating a pathologic condition associated with LAG-3 expression, overexpression or activity, comprising administering to a subject in need thereof, a pharmaceutical composition comprising a therapeutically effective amount of the antibody or antibody fragment thereof described herein.

According to some embodiments, the LAG-3 ligand is influenza HA. According to other embodiments, the LAG-3 ligand is a lectin. According to some specific embodiments, the lectin is a human C-type lectin (LSECtin/CLEC4G) or human Galectin3 (Gal-3). According to some further embodiments, the LAG-3 ligand is a human MHC class II molecule. According to yet further embodiments, the LAG-3 ligand is a human Fibrinogen-like protein 1 (FGL1). According to further embodiments, the LAG-3 ligand is a human Alpha-synuclein pff (α-Syn PFF).

According to some embodiments, the disease or disorder associated with human LAG-3 expression is selected from the group including but not limited to: influenza, cancer, autoimmune disease, infectious disease and neurogenerative disease.

According to some embodiments, the neurogenerative disease is a CNS disease selected from Parkinson's disease and Alzheimer's disease.

The present invention provides, according to some embodiments, a method of preventing, inhibiting, delaying or treating a malignancy, a cancer or a tumor comprising administering to a subject in need thereof, a pharmaceutical composition comprising a therapeutically effective amount of the antibody or antibody fragment thereof described herein.

According to some embodiments of the invention, the cancer is selected from the group consisting of a lung cancer, a breast cancer, a colorectal cancer, a melanoma, an ovarian cancer, a pancreatic cancer, a colon cancer, a cervical cancer, a kidney cancer, a thyroid cancer, a prostate cancer, a brain cancer, a renal cancer, a throat cancer, a laryngeal carcinoma, a bladder cancer, a hepatic cancer, a fibrosarcoma, an endometrial cells cancer, a glioblastoma, and sarcoma. Each possibility represents a separate embodiment of the invention. According to some embodiments, the cancer or tumor is selected from the group consisting of Melanoma, Hodgkin's lymphoma, Diffuse large B-cell lymphoma, Urothelial cancer, Colorectal cancer, Hepatocellular carcinoma, Non-small cell lung cancer, Small cell lung cancer, Renal cell carcinoma, Squamous cell carcinoma, Gastric cancer, Esophageal cancer, Cutaneous squamous cell carcinoma, Triple negative breast cancer, and Merkel cell carcinoma. Each possibility represents a separate embodiment of the invention.

According to some embodiments, the cancer is a solid cancer or comprises a solid tumor. According to some specific embodiments, the cancer is selected from the group consisting of breast cancer, colorectal cancer, lung cancer, bladder cancer, pancreatic cancer, ovarian cancer, kidney cancer, melanoma, prostate cancer, and brain cancer. Each possibility represents a separate embodiment of the invention.

According to some embodiments, the cancer or tumor is selected from the group consisting of renal cancer, ovarian cancer, colon cancer, prostate cancer, and breast cancer. According to some embodiments, the cancer or tumor is selected from the group consisting of renal cancer, colon cancer, prostate cancer and breast cancer. Each possibility represents a separate embodiment of the invention.

According to other embodiments, the cancer is hematologic cancer selected from a myeloma, a leukemia, and a lymphoma.

According to some embodiments, the cancer is a metastatic cancer.

According to some embodiments, the method involves or results in inhibiting a metastatic cascade, namely preventing, reducing or inhibiting metastases formation, migration, adhesion, spread, and/or growth.

According to some embodiments, the method comprises administering the pharmaceutical composition comprising the antibody of the invention, before, together with or following treatment. According to some embodiments, the treatment is selected from surgery, chemotherapy, radiotherapy, immunotherapy, and any combinations thereof.

According to some embodiments of the invention, the treatment results in a decrease in tumor size or in the number, size or spread of metastases in the subject.

According to some embodiments, the method of treating cancer comprises administering or performing at least one additional anti-cancer therapy or treatment. According to certain embodiments, the additional anticancer therapy is surgery, chemotherapy, radiotherapy, or immunotherapy.

According to some embodiments, the method of treating cancer comprises administration of the antibody and an additional anti-cancer agent. According to some embodiments, the additional anti-cancer agent is selected from the group consisting of: an immune-modulator, an agent that inhibits immune co-inhibitory receptor, activated lymphocyte cells, a kinase inhibitor, and a chemotherapeutic agent.

According to some embodiments, the additional immune-modulator is an antibody against an immune checkpoint molecule. According to some embodiments, the additional immune modulator is an antibody against an immune checkpoint molecule selected from the group consisting of human programmed cell death protein 1 (PD-1), PD-L1 and PD-L2, carcinoembryonic antigen-related cell adhesion molecule 1 (CEACAM1), CD137, OX40 (also referred to as CD134), killer cell immunoglobulin-like receptors (KIR), TIGIT, PVR, CTLA-4, NKG2A, GITR, and any other checkpoint molecule or a combination thereof. Each possibility represents a separate embodiment of the invention.

According to some embodiments, the anti-cancer agent is selected from the group consisting of: erbitux, cytarabine, fludarabine, fluorouracil, mercaptopurine, methotrexate, thioguanine, gemcitabine, vincristine, vinblastine, vinorelbine, carmustine, lomustine, chlorambucil, cyclophosphamide, cisplatin, carboplatin, ifosfamide, mechlorethamine, melphalan, thiotepa, dacarbazine, bleomycin, dactinomycin, daunorubicin, doxorubicin, idarubicin, mitomycin, mitoxantrone, plicamycin, etoposide, teniposide, paclitaxel, and any combination thereof. Each possibility represents a separate embodiment of the invention.

According to some embodiments of the invention, the subject is a human subject.

Any administration route suitable for delivery of proteins or antibodies may be used with the compositions and methods of the present invention and the compositions administered are formulated according to the administration mode. According to some embodiments, the antibody is administered parenterally. According to some embodiments, the antibody is administered via a route selected from intravenously, intramuscularly, subcutaneously, intra-tumorally, intra-dermally, intra-arterially, intraarticularly, intralesionally or submucosally, intranasally, orally, and topically.

Ordinarily, intravenous (i.v.) administration by infusion or injection is used. In other embodiments, the composition is administered via an intra-tumoral route. In other embodiments, the composition is administered during or following surgery.

Also provided, according to another aspect of the invention is a method of delivering an antibody to human LAG-3 disclosed herein or an antibody fragment thereof to a cell, the method comprising contacting the cell with the at least one antibody, or antibody fragment.

According to some embodiments, the cell is of a human subject.

The present invention further comprises, according to another aspect, a method of determining or quantifying human LAG-3 in a sample, the method comprising contacting a biological sample with an antibody or antibody fragment described above and measuring the level of complex formation.

According to some embodiments, the method for detecting or quantifying the expression of human LAG-3 comprises the steps of:
i. incubating a sample with an antibody specific to human LAG-3 or an antibody fragment thereof comprising at least an antigen-binding portion; and
ii. detecting the bound LAG-3 using a detectable probe.

According to some embodiments, the method further comprises the steps of:
iii. comparing the amount of (ii) to a standard curve obtained from a reference or a control sample containing a known amount of LAG-3; and
iv. calculating the amount of the LAG-3 in the sample from the standard curve.

According to some embodiments, the method comprises comparing the binding of a mAb according to the present invention to pathological tissue or cells, with the binding to normal tissue or cells.

The antibodies according to the present invention may also be used to configure screening methods. For example, an enzyme-linked immunosorbent assay (ELISA), or a radioimmunoassay (RIA), as well as methods such as immunohistochemistry (IHC) or fluorescence-activated cell sorting (FACS), can be constructed for measuring levels of secreted or cell-associated LAG-3 in a biological sample, using the antibodies and methods known in the art.

According to some embodiments, the biological sample is a body fluid or tissue.

According to some embodiments, the method is performed in-vitro or ex-vivo.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The novel features described herein are set forth with particularity in the appended claims. A better understanding of the characteristics and advantages of the features described herein will be obtained by reference to the following detailed description that sets forth illustrative examples, in which the principles of the features described herein are utilized, and the accompanying drawings of which:

FIGS. 1A-1D present the results of staining experiments of influenza-virus infected cells with LAG-3. FIGS. 1A-1B show A549 cells infected with A/Puerto Rico/8/1934 (H1N1) and stained 48 hours post infection with anti-HA monoclonal antibody and with hLAG-3-Ig, respectively. Uninfected A549 cells served as a negative control. The grey filled histogram represents uninfected cells, and the black empty histogram represents infected cells. Shown is one representative experiment out of three performed. FIG. 1C shows a summary of the mean fluorescent intensity (MFI) seen in staining of uninfected and influenza infected A549 cells (statistically significant differences are shown). FIG. 1D shows A549 cells were infected with A/Puerto Rico/8/1934 (HIN1) and were 48 hours post infection stained with mLAG-3-Ig. Uninfected A549 cells were used as control. The grey filled histogram represents uninfected cells, and the black empty histogram represents infected cells.

FIGS. 2A-2E present the results of the binding assays of LAG-3 with HA glycoprotein. FIGS. 2A-2B show ELISA plates coated with hLAG3-Ig and incubated with HA glycoprotein of A/Puerto Rico/8/1934 (H1N1) (right column), SARS-COV-2 spike glycoprotein (FIG. 2B, middle column) and with PBSX1 (control, left column), respectively. LAG-3-HA and LAG-3-SARS-COV-2 interactions were determined using anti-HA and anti-SARS-Cov-2 respective monoclonal antibodies. Shown are mean values and standard errors of the optical density (OD 450) from three experiments, including statistically significant differences. FIGS. 2C-2D show staining with anti-HA antibody and with hLAG3-Ig, respectively, of MDCK cells engineered to express HA glycoprotein of A/Puerto Rico/8/1934 (HINI). Parental/native MDCK cells served as a negative control. The grey filled histogram represents parental/native MDCK cells, and the black empty histogram represents engineered MDCK cells. Shown is one representative experiment out of three performed. FIG. 2E show the binding of mouse LAG-3 to purified HA. ELISA plates were coated with mouse LAG-3 and, after blocking, were incubated with H1N1 PR8 HA protein-HIS tag. Samples were then stained with anti-HIS tag antibody. Wells without mouse LAG-3 were used as negative control. Shown are mean values and standard errors from four independent experiments, including statistically significant differences (T-test).

FIG. 3 shows the results of an ELISA, measuring the binding of the influenza-virus HA to alternative proteins respectively coated on the ELISA plates. A hLAG-3-Ig coated plate served as a positive control, a hLAG-3-Ig coated plate incubated without HA served as a negative control. The name of the proteins is depicted in the X axis, and the Y axis depicts OD 650. Significant changes are shown.

FIGS. 4A-4B show the binding of LAG-3 to the HA. FIG. 4A depicts the results of an ELISA measuring the binding of LAG-3 to the HA of three influenza streams, influenza A H1N1 (A/Puerto Rico/8/1934), influenza A H5N1 (A/Vietnam/1194/2004), and influenza A H3N2 (A/Wisconsin/67/2005). The name of the proteins is depicted in the X axis, and the Y axis depicts OD 650. FIG. 4B shows the binding kinetics between hLAG-3 to HA.

FIG. 5 is a schematic representation of LAG-3 structure and the location of N-linked glycans.

FIGS. 6A-6B present the results of binding analysis of glycan-free LAG-3 with HA. FIG. 6A shows SDS-PAGE analysis of LAG-3-Ig, and LAG-3-Ig treated with PNGase F. FIG. 6B shows the result of an ELISA, measuring the binding of HA with LAG-3-Ig coated plates, and with PNGase F-treated LAG-3-Ig coated plates, respectively. A hLAG-3-Ig coated ELISA plate incubated without HA served as a negative control. Significant changes are shown.

FIG. 7 shows the binding of uninfected and influenza virus infected A549 cells with the various LAG-3 domains (D1, D2, D3 and D4).

FIGS. 8A-8B portray the experiment and results of the interaction between human LAG-3 D2 domain and HA. FIG. 8A is a schematic representation of the different stages of the experiment. FIG. 8B shows the results of an ELISA, measuring the binding of HA with LAG-3D2-Fc coated plates. The LAG-3D2-Fc coated plates were first incubated with serum from mice immunized with full length hLAG-3 (polyclonal antibodies), washed and then added with influenza virus HA protein to each well.

FIGS. 9A-9B represent the experiment and results of the effect of influenza virus HA glycoprotein on T-cell activation. FIG. 9A is a schematic representation of the experiment.

FIG. 9B shows a flow cytometry results of the influenza virus HA glycoprotein effect on T-cell activation (percentage of CD8+ Cd25+ cells).

FIGS. 10A-10C show mLAG-3 expression on T-cell post influenza virus infection in vivo. FIG. 10A displays the weight of H1N1 influenza virus infected and uninfected mice.

FIGS. 10B-10C show LAG-3-positive CD8+ T cells as measured in the lungs of infected mice harvested on days 3, 5, 7 and 10. FIG. 10B shows the percentage of LAG-3-positive CD8+ T cells. FIG. 8C shows the surface expression level of LAG-3 on CD8+ T cells that were isolated from the infected mice.

FIGS. 11A-11C represent the results of LAG-3 treatment to Influenza virus-infected mice, as measured by lung T-cell activity. FIG. 11A is a schematic representation of the different stages of the experiment. FIG. 11B shows the gating strategy performed on the CD8 T cells derived from mice lungs, including FSC/SSC measurement, viability measurement, and CD8/CD3 marker recognition. One representative experiment is depicted out of three performed. FIG. 11C shows the fold increase in the percentage, out of all the CD8 positive T cells, of CD69-CD8 positive T cells and CD25-CD8 positive T cells, respectively, isolated from the lungs of infected untreated mice and LAG-3-Ig-treated mice, respectively, as compared with uninfected mice. The mean values from each mice group are shown. The mean percentage of the CD69-CD8 positive T cells and CD25-CD8 positive T cells from the lungs of uninfected mice was set at 1.

### DETAILED DESCRIPTION OF THE INVENTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the term “individual,” “patient,” or “subject” refers to individuals diagnosed with, suspected of being afflicted with, or at-risk of developing at least one disease for which the described compositions and method are useful for treating. According to some embodiments, the individual is a mammal. According to some embodiments, the mammal is a mouse, rat, rabbit, dog, cat, horse, cow, sheep, pig, goat, llama, alpaca, or yak. According to some embodiments, the individual is a human.

As used herein the term “combination” or “combination treatment” can refer either to concurrent administration of the articles to be combined or sequential administration of the articles to be combined. As described herein, when the combination refers to sequential administration of the articles, the articles can be administered in any temporal order.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, or, in cases of autoimmunity or pathological inflammation, normal human cells or tissues.

### Antibodies and Polypeptides

The term “antibody” herein is used in the broadest sense and includes polyclonal and monoclonal antibodies, including intact antibodies and functional (antigen-binding) antibody fragments thereof, including fragment antigen-binding (Fab) fragments, F(ab′)2 fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, fully human antibodies, humanized antibodies, and heteroconjugate antibodies, multispecific, e.g., bi-specific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

An “antigen-specific T cell response” refers to responses by a T cell that result from stimulation of the T cell with the antigen for which the T cell is specific. Non-limiting examples of responses by a T cell upon antigen-specific stimulation include proliferation and cytokine production (e.g., IL-2 production).

The term “influenza infection”, as used herein, also characterized as “flu” refers to the severe acute respiratory illness caused by influenza virus. The term includes respiratory tract infection and the symptoms that include high fever, headache, general aches and pains, fatigue and weakness, in some instances extreme exhaustion, stuffy nose, sneezing, sore throat, chest discomfort, cough, shortness of breath, bronchitis, pneumonia and death in severe cases.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modeling antibodies on the WEB,” Protein Eng. 2000 December; 13 (12): 819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments.

By the term “single-chain variable fragment (scFv)” is meant a fusion of the V_{H} and V_{L} regions, linked together with a short (usually serine, glycine) linker. Single-chain antibodies can be single chain composite polypeptides having antigen binding capabilities and comprising amino acid sequences homologous or analogous to V_{H} and V_{L} VL (linked V_{H}-V_{L} or a single chain Fv (scFv)). Both V_{H} and V_{L} may copy natural mAb sequences or one or both of the chains may comprise a CDR-FR construct of the type described in U.S. Pat. No. 5,091,513. The separate polypeptides analogous to the V_{H} and V_{L} regions are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. According to some embodiments, the antibody fragments are scFvs.

The term “antigen” as used herein refers to a molecule or a portion of a molecule capable of eliciting antibody formation and being specifically bound by a binding molecule such as an antibody or a fragment thereof comprising the antigen-binding site. An antigen may have one or more than one epitope. The specific binding referred to above is meant to indicate that the antigen will react, in a highly selective manner, with its corresponding antibody and not with the multitude of other antibodies which may be evoked by other antigens. According to some embodiments of the present invention, at least one of the N-glycosylation sites of the LAG-3 glycoprotein are the antigenic targets of the anti-LAG-3 monoclonal antibodies.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. According to some embodiments, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN or Megalign (DNASTAR) software. Appropriate parameters for aligning sequences can be determined, including algorithms needed to achieve maximal alignment over the full length of the sequences being compared.

The terms “homologous”, “homology” or “percent homology” when used herein to describe an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, for example.

In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion and substitution can be made to arrive at the final construct, provided that the final construct possesses the desired characteristics, e.g., antigen-binding.

In some embodiments, antibody variants having one or more amino acid substitutions are provided. Sites of interest for mutagenesis by substitution include the CDRs and FRs. Amino acid substitutions may be introduced into an antibody of interest and the products screened for a desired activity, e.g., retained/improved antigen binding, decreased immunogenicity, or improved antibody-dependent cellular cytotoxicity (ADCC) or complement-dependent cytotoxicity (CDC).

In some embodiments, substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments of the variant V_{H} and V_{L} sequences, each CDR is unaltered.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3, in particular, are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

“Specific binding” or “specifically binds” or “binds” refer to an antibody binding to a specific antigen with greater affinity than for other antigens. Typically, the antibody “specifically binds” when the equilibrium dissociation constant (K_{D}) for binding is about 1×10⁻⁷ M or less, for example about 1×10⁻⁸ M or less, about 1×10⁻⁹ M or less, about 1×10⁻¹⁰ M or less, or about 1×10⁻¹² M or less, typically with the KD that is at least one hundred-fold less than its KD for binding to a non-specific antigen (e.g, BSA, casein). The K_{D} may be measured using standard procedures. In some embodiments, an antibody, fusion protein, or other molecule provided herein that specifically inhibits the binding of influenza hemagglutinin (HA) to mammalian Lymphocyte activation gene 3 (herein also “LAG-3-HA binding inhibitor”) may be further modified to contain additional non-proteinaceous moieties that are known and available. The moieties suitable for derivatization of the antibody include but are not limited to water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone) polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the construct may vary, and if two or more polymers are attached, they can be the same or different molecules.

The antibody fragments, conjugates and constructs described herein can be encoded by a nucleic acid. A nucleic acid is a type of polynucleotide comprising two or more nucleotide bases. In certain embodiments, the nucleic acid is a component of a vector that can be used to transfer the polypeptide-encoding polynucleotide into a cell. As used herein, the term “vector” refers to a nucleic acid molecule capable of transporting another nucleic acid to which it has been linked. One type of vector is a genomically integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors, regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

Any human IgG molecule or a portion thereof may be used according to the present invention as a carrier for the antigen-binding molecule. According to some embodiments, the human IgG is selected from IgG1, IgG2, IgG3 and IgG4 or portions thereof. According to some embodiments, the carrier molecule is a human IgG1 constant region or a potion thereof.

In some embodiments, one or more amino acid modifications may be introduced into the Fc region of a construct provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single-stranded or double-stranded.

“Expression vector” refers to a vector that can be utilized in a biological system or in a reconstituted biological system to direct the translation of a polypeptide encoded by a polynucleotide sequence present in the expression vector.

As used herein, the term “heterologous” used in reference to nucleic acid sequences, proteins or polypeptides, means that these molecules are not naturally occurring in the cell from which the heterologous nucleic acid sequence, protein or polypeptide was derived. For example, the nucleic acid sequence coding for a human polypeptide that is inserted into a cell that is not human is a heterologous nucleic acid sequence in that particular context. Whereas heterologous nucleic acids may be derived from a different organism or animal species, such nucleic acid need not be derived from separate organism species to be heterologous. For example, in some instances, a synthetic nucleic acid sequence or a polypeptide encoded therefrom may be heterologous to a cell into which it is introduced in that the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

A “host cell,” as used herein, denotes an in vivo or in vitro eukaryotic cell or a cell from a multicellular organism (e.g., a cell line) cultured as a unicellular entity, which eukaryotic cells can be, or have been, used as recipients for nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g, an exogenous nucleic acid that is foreign to the eukaryotic host cell, or a recombinant nucleic acid that is not normally found in the eukaryotic host cell.

### Lymphocyte Activation Gene-3 (LAG-3)

The term “LAG-3” refers to Lymphocyte Activation Gene-3. The LAG-3 protein, which belongs to immunoglobulin (Ig) superfamily, comprises a 503-amino acid type I transmembrane protein with four extracellular Ig-like domains, designated D1 to D4 (corresponding to SEQ ID Nos. 6, 7, 8 and 9 respectively). As described herein, the term “LAG-3” includes variants, isoforms, homologs, orthologs, and paralogs. For example, antibodies specific for a human LAG-3 protein may, in certain cases, cross-react with a LAG-3 protein from a species other than human. In other embodiments, the antibodies specific for a human LAG-3 protein may be exclusive the human LAG-3 protein and may not exhibit any type of cross-reactivity. The term “human LAG-3” refers to human sequence LAG-3, such as the complete amino acid sequence of human LAG-3 having GenBank Accession No. NP 002277:

(SEQ ID NO. 1)
MWEAQFLGLLFLQPLWVAPVKPLQPGAEVPVVWAQEGAPAQLPCSPTIPL
QDLSLLRRAGVTWQHQPDSGPPAAAPGHPLAPGPHPAAPSSWGPRPRRYT
VLSVGPGGLRSGRLPLQPRVQLDERGRQRGDFSLWLRPARRADAGEYRAA
VHLRDRALSCRLRLRLGQASMTASPPGSLRASDWVILNCSFSRPDRPASV
HWFRNRGQGRVPVRESPHHHLAESFLFLPQVSPMDSGPWGCILTYRDGFN
VSIMYNLTVLGLEPPTPLTVYAGAGSRVGLPCRLPAGVGTRSFLTAKWTP
PGGGPDLLVTGDNGDFTLRLEDVSQAQAGTYTCHIHLQEQQLNATVTLAI
ITVTPKSFGSPGSLGKLLCEVTPVSGQERFVWSSLDTPSQRSFSGPWLEA
QEAQLLSQPWQCQLYQGERLLGAAVYFTELSSPGAQRSGRAPGALPAGHL
LLFLILGVLSLLLLVTGAFGFHLWRRQWRPRRFSALEQGIHPPQAQSKIE
ELEQEPEPEPEPEPEPEPEPEPEQL.

LAG-3 is also known in the art as, for example, CD223. The human LAG-3 sequence may differ from human LAG-3 of GenBank Accession No. NP 002277 by having, e.g., conserved mutations or mutations in non-conserved regions, while maintaining substantially the same biological function as the human LAG-3 of GenBank Accession No. NP 002277, particularly in the context of LAG-3-HA binding domains.

A particular human LAG-3 sequence will generally be at least 90% identical in amino acids sequence to human LAG-3 of GenBank Accession No. NP 002277 and contains amino acid residues that identify the amino acid sequence as being human when compared to LAG-3 amino acid sequences of other species (e.g., murine). In certain cases, a human LAG-3 can be at least 95%, or even at least 96%, 97%, 98%, or 99% identical in amino acid sequence to LAG-3 of GenBank Accession No. NP 002277. In certain embodiments, a human LAG-3 sequence will display no more than 40 amino acids differences from the LAG-3 sequence of GenBank Accession No. NP 002277. In certain embodiments, the human LAG-3 can display no more than 20, 10, 5, or even no more than 4, 3, 2, or 1 amino acid difference from the LAG-3 sequence of GenBank Accession No. NP 002277.

As used herein, the term “LAG-3 fragment, conjugate, and/or fusion protein” refers to a mammalian LAG-3 protein, or a fragment thereof, which inhibits the interaction between LAG-3 glycoprotein which is displayed upon cells of the immune system, in particular CD8 T cells, and viral HA. According to some embodiments the LAG-3 fragment is fused to at least one polypeptide selected from: a carrier polypeptide, human immunoglobulin, and albumin. According to some embodiments the LAG-3 fragment, conjugate and/or fusion protein is fused to the polypeptide via a linker. According to other embodiments the LAG-3 fragment is directly fused to the polypeptide. According to some particular embodiments the LAG-3 fragment is fused to IgG1 Fc constant region. According to some exemplary embodiments the LAG-3 fragment includes the following amino-acid sequence (amino acids 23-450 from SEQ ID NO. 1):

(SEQ ID NO. 2)
LQPGAEVPVVWAQEGAPAQLPCSPTIPLQDLSLLRRAGVTWQHQPDSGPP
AAAPGHPLAPGPHPAAPSSWGPRPRRYTVLSVGPGGLRSGRLPLQPRVQL
DERGRQRGDFSLWLRPARRADAGEYRAAVHLRDRALSCRLRLRLGQASMT
ASPPGSLRASDWVILNCSFSRPDRPASVHWFRNRGQGRVPVRESPHHHLA
ESFLFLPQVSPMDSGPWGCILTYRDGFNVSIMYNLTVLGLEPPTPLTVYA
GAGSRVGLPCRLPAGVGTRSFLTAKWTPPGGGPDLLVTGDNGDFTLRLED
VSQAQAGTYTCHIHLQEQQLNATVTLAIITVTPKSFGSPGSLGKLLCEVT
PVSGQERFVWSSLDTPSQRSFSGPWLEAQEAQLLSQPWQCQLYQGERLLG
AAVYFTELSSPGAQRSGRAPGALPAGHL.

According to some exemplary embodiments the IgG1 Fc constant region comprises the following amino-acid sequence:

(SEQ ID NO. 3)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

According to some exemplary embodiments the conjugate, including the fragment of LAG-3 fused to IgG1 Fc constant region, comprises the following amino-acid sequence:

(SEQ ID NO. 4)
LQPGAEVPVVWAQEGAPAQLPCSPTIPLQDLSLLRRAGVTWQHQPDSGPP
AAAPGHPLAPGPHPAAPSSWGPRPRRYTVLSVGPGGLRSGRLPLQPRVQL
DERGRQRGDFSLWLRPARRADAGEYRAAVHLRDRALSCRLRLRLGQASMT
ASPPGSLRASDWVILNCSFSRPDRPASVHWFRNRGQGRVPVRESPHHHLA
ESFLFLPQVSPMDSGPWGCILTYRDGFNVSIMYNLTVLGLEPPTPLTVYA
GAGSRVGLPCRLPAGVGTRSFLTAKWTPPGGGPDLLVTGDNGDFTLRLED
VSQAQAGTYTCHIHLQEQQLNATVTLAIITVTPKSFGSPGSLGKLLCEVT
PVSGQERFVWSSLDTPSQRSFSGPWLEAQEAQLLSQPWQCQLYQGERLLG
AAVYFTELSSPGAQRSGRAPGALPAGHLASTKGPSVFPLAPSSKSTSGGT
AALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQK
SLSLSPGK.

According to some embodiments the LAG-3 fragment, conjugate and/or fusion protein may include a sequence corresponding to a portion of the human LAG-3 protein. According to some embodiments the sequence may correspond to a portion of LAG-3 protein spanning a length of at least 20 amino acids. According to further embodiments the sequence may correspond to a portion of LAG-3 protein spanning a length of at least 30 amino acids, at least 50 amino acids, at least 100 amino acids, at least 150 amino acids, at least 200 amino acids, at least 250 amino acids, at least 300 amino acids, at least 350 amino acids, at least 400 amino acids, at least 450 amino acids, or at least 500 amino acids. Each possibility represents a separate embodiment of the invention. According to some embodiments, the LAG-3 fragment, conjugate, or fusion protein includes at least one of the LAG-3 N-glycosylation sites. The LAG-3 N-glycosylation sites include amino acid 188, amino acid 250, amino acid 256, and amino acid 343, of the LAG-3 sequence (SEQ ID NO. 1).

According to some embodiments the LAG-3 fragment, conjugate, and/or fusion protein is externally introduced into the treated subject, e.g., into an infected tissue thereof. According to further embodiments a nucleic acid sequence which encodes the free/externally introduced LAG-3 is introduced into the subject, and the free/externally introduced LAG-3 protein is subsequently produced by the subject.

### Hemagglutinin (HA) Polypeptides and Influenza Strains

Influenza viruses are negative sense, single-stranded, segmented RNA envelope viruses. Two glycoproteins, a hemagglutinin (HA) polypeptide and a neuraminidase (NA) polypeptide, are displayed on the outer surface of the viral envelope. As mentioned above, HA is the major viral surface glycoprotein that mediates binding and entry of the virus into host cells and is a primary target of neutralizing antibody responses. HA is a trimer of three identical monomers. Each monomer is synthesized as a precursor, HA0, that is proteolytically processed into two disulfide-bonded polypeptide chains, HA1 and HA2. The ectodomain of this protein has (i) a globular head domain, possessing receptor binding activity and major antigenic determinants, (ii) a hinge region, and (iii) a stem region where a sequence critical for the fusion of the viral envelope and the cell wall (the “fusion peptide”) is located. The viral replication cycle is initiated when the virion attaches via its surface hemagglutinin proteins to sialylated glycan receptors on the host cell and enters the cell by endocytosis. The acidic environment in the endosome induces conformational changes in HA that expose the fusion peptide hidden within the stem region of the trimer. The exposed fusion peptide mediates the fusion of the viral and target cell membranes resulting in the release of the viral ribonucleoprotein into the cell cytoplasm.

According to some embodiments of the present invention, the term “influenza” refers to at least one strain of influenza A and/or influenza B. Influenza A viruses are divided into subtypes, labeled according to an H number (for the type of hemagglutinin) and an N number (for the type of neuraminidase). There are 18 different H antigens (H1 to H18) and 11 different N antigens (N1 to N11). Influenza A strains are identified by a nomenclature based on the number of the strain's HA polypeptide and NA polypeptide subtypes, for example, H1N1, HIN2, H1N3, H1N4, H1N5, and the like. Influenza B virus, on the other hand, is divided into lineages rather than subtypes, which lineages differ based on antigenic properties of their respective surface HA proteins. As influenza A can infect and be carried by a variety of animal species, it is commonly transferred to human beings from other animals, particularly birds. By contrast, Influenza B is known to infect only human beings and seals, and is therefore transferred only from human to human.

While more than 130 influenza A subtype combinations have so far been identified in nature (primarily originating from wild birds), there are potentially many more influenza A subtype combinations, given the propensity for virus “reassortment”. Reassortment is a process by which influenza viruses swap gene segments and can occur when two influenza viruses infect a host concurrently and swap genetic information. Some examples of subtypes of influenza A viruses that routinely circulate amongst the human population include, but are not limited to, H3N2, seasonal H1N1, and 2009 H1N1. As for influenza B, two lineages are known to currently circulate, termed B/Yamagata/16/88-like and B/Victoria/2/87-like viruses. Influenza A subtypes can be further broken down into different genetic “clades” and “sub-clades” according to their genetic and antigenic properties. This division into clades and sub-clades may assist in predicting the effectivity of an innate immune response, vaccine, or antibody, which were developed against a particular strain, in protecting against a different influenza virus strain.

### Compositions and Therapeutic Methods

Compositions and methods are disclosed herein for the prevention or treatment of an influenza virus infection. Prevention can include inhibition of infection with influenza. Treatment includes diminishing signs and symptoms of an influenza virus infection and/or reducing viral titer. The methods include contacting a cell with an effective amount of the LAG-3-HA binding inhibitor disclosed herein. The LAG-3-HA binding inhibitor may include, for example, monoclonal antibodies that specifically bind LAG-3, or a LAG-3 fragment, conjugate, or fusion protein (e.g., a LAG-3-Ig conjugate) which binds HA and prevents its binding to human LAG-3 present in cells. Any other molecules which prevent or reduce the binding of viral HA to human LAG-3 may also serve as LAG-3-HA binding inhibitor. The method can also include administering to a subject a therapeutically effective amount of a LAG-3-HA binding inhibitor, or a nucleic acid encoding the LAG-3-HA binding inhibitor. According to some particular embodiments, the anti-LAG-3 monoclonal antibody binds at least one N-glycosylation site of LAG-3.

The treated subject can be a human or a veterinary subject. According to some embodiments, the treated subject may be a population or a group of subjects. In some embodiments, a population of subjects is being exposed to influenza virus under pandemic conditions. Humans at high risk of infection, such as immunocompromised individuals, and humans who are at high risk of exposure to influenza virus may be particularly suited to receive treatment with the LAG-3-HA binding inhibitor molecule. Immunocompromised individuals include the elderly (65 years and older) and children (e.g., 6 months to 18 years old), and people with chronic medical conditions. People at high risk of exposure include health care workers, teachers and emergency responders (e.g., firefighters, policemen). According to one embodiment, the subject is hospitalized. According to another embodiment, the subject is not hospitalized.

Methods are disclosed herein for reducing the risk of infection with influenza virus in a human subject, particularly of influenza A and/or B, the method including administering the LAG-3-HA binding inhibitor. Methods are also disclosed for preventing influenza disease in a subject, the method including administering the LAG-3-HA binding inhibitor.

Methods are disclosed herein for treating a subject infected with influenza virus, the method including administering the LAG-3-HA binding inhibitor. Methods are also disclosed for ameliorating one or more symptoms associated with influenza infection in a subject, the method including administering the LAG-3-HA binding inhibitor.

Influenza virus infection does not need to be eliminated for the composition to be effective. For example, a composition can decrease influenza infection in a population by a desired amount, for example by at least 10%, at least 20%, at least 50%, at least 60%, at least 70%, at least 80%, at least 90%, at least 95%, at least 98%, or even at least 100%, as compared to the rate of infection in the absence of the composition.

According to some embodiments, the anti-influenza LAG-3-HA binding inhibitor and/or compositions including one or more of the LAG-3-HA binding inhibitors can be administered for prevention and/or treatment of influenza disease caused by an influenza A virus. In certain non-limiting embodiments, the anti-influenza LAG-3-HA binding inhibitor can be administered for prevention and/or treatment of influenza disease caused by an influenza A H1N1, influenza A H5N1 and/or influenza A H3N2 infection.

Compositions are provided that include one or more of the LAG-3-HA binding inhibitors according to the present invention, or therapeutically effective fragments thereof (i.e., therapeutically effective fragment of a LAG-3 binding antibody according to the present invention, a therapeutically effective fragment of a LAG-3-Ig conjugate or other LAG-3 fusion protein, and the like) and nucleic acids encoding the LAG-3-HA binding inhibitors (or fragments thereof) that are disclosed herein in a carrier. The compositions may be prepared in unit dosage forms for administration to a subject. The amount and timing of administration are at the discretion of the treating physician to achieve the desired purposes. The composition containing LAG-3-HA binding inhibitor can be formulated for systemic or local administration. According to some embodiments, the composition containing LAG-3-HA binding inhibitor is formulated for parenteral administration, such as intravenous injection or infusion, subcutaneous injection, or intramuscular injection. The LAG-3-HA binding inhibitor can be administered as a fixed dose, or in a mg/kg dose. The compositions for administration may include a solution of the LAG-3-HA binding inhibitor dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of LAG-3-HA binding inhibitor in these formulations can vary widely, and may be selected based on fluid volumes, viscosities, body weight and the like in accordance with the particular mode of administration selected and the subject's needs.

Dosages from 0.1 up to about 100 mg per subject per day may be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Science, 19th ed., Mack Publishing Company, Easton, Pa. (1995).

According to some embodiments, LAG-3-HA binding inhibitor may be provided in lyophilized form and rehydrated with sterile water before administration, or in sterile solutions of known concentration. According to some embodiments, LAG-3-HA binding inhibitor may be administered by slow infusion, by an intravenous push or bolus. In one example, a higher loading dose is administered, with subsequent, maintenance doses being administered at a lower level.

According to some non-limiting exemplary embodiments, LAG-3-HA binding inhibitor as in the present invention can be administered intravenously. According to some embodiments, LAG-3-HA binding inhibitor can be administered subcutaneously. According to some embodiments, a LAG-3-HA binding inhibitor can be administered by inhalation, such as by intranasal or by oral inhalation. According to some further embodiments, a LAG-3-HA binding inhibitor can be administered in a bolus. Any other delivery systems and/or modules as are known in the art may be utilized for administering of the LAG-3-HA binding inhibitor. In some non-limiting embodiments, the binding agent, e.g., an anti-LAG-3 antibody molecule, may be administered buccally, orally, by nasal delivery (e.g., as a liquid, spray, aerosol), by topical application, (e.g., as a gel, cream, liquid, drops, or emulsion) or by inhalation.

According to some embodiments the LAG-3-HA binding inhibitor may be administered at a fixed unit dose, in the range of between about 50 mg and about 5000 mg, e.g., between 50 mg and 500 mg, between 50 mg and 2500 mg, between 100 mg and 1000 mg, between 100 mg and 3000 mg, between 300 mg and 1500 mg, between 500 mg and 2000 mg, between 500 mg and 5000 mg, between 1000 mg and 2500 mg, between 1500 mg and 3000 mg, between 1500 mg and 4000 mg, between 2000 mg and 4000, between about 2000 mg and about 5000 mg, between 3000 mg and 4000, or between 3000 mg and 5000, including each value within each of the specified ranges. Each possibility represents a separate embodiment.

According to some embodiments the LAG-3-HA binding inhibitor may be administered at a fixed unit dose of about 50 mg, 60 mg, 70 mg, 75 mg, 80 mg, 100 mg, or about 120 mg, 130 mg, 140 mg, 150 mg, 160 mg, 180 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, about 2500 mg, or more. Each possibility represents a separate embodiment.

According to some further embodiments, a LAG-3-HA binding inhibitor can be administered at a dose which is dependent in the weight of the subject. As a non-limiting example, a weigh-dependent dose may be in the range of about 0.5 and about 50 mg/kg of subject weight, e.g., about 0.5 and 5 mg/kg, 1 and 10 mg/kg, about 1 and 25 mg/kg, about 5 and 50 mg/kg, about 10 and 50 mg/kg, or about 25 and 50 mg/kg, including each value within each of the specified ranges. Each possibility represents a separate embodiment. Weight dependent administration may be limited so as not to exceed a certain dosage per subject. As a non-limiting example the dose may be limited to is less than about 10000 mg/subject, about 8000 mg/subject, about 5000 mg/subject, about 3000 mg/subject, about 1500 mg/subject, about 1000 mg/subject, about 600 mg/subject, about 500 mg/subject, about 400 mg/subject, about 300 mg/subject, about 250 mg/subject, about 200 mg/subject, about 150 mg/subject, or about 100 mg/subject. Each possibility represents a separate embodiment.

Dosing can be adjusted according to a patient's rate of clearance of a prior administration of the LAG-3-HA binding inhibitor. For example, a patient may not be administered a second or follow-on dose before the level of antibodies in the patient's system has dropped below a pre-determined level. In one embodiment, a sample from a patient (e.g., plasma, serum, blood, urine, or cerebrospinal fluid (CSF)) is assayed for the presence of a LAG-3-HA binding inhibitor according to the present invention, and if the level thereof is above a pre-determined level, the patient will not be administered a second or follow-on dose. If the level of LAG-3-HA binding inhibitors in the patient's system drops below a pre-determined level, then the patient may be administered a second or follow-on dose. A patient whose LAG-3-HA binding inhibitor levels are determined to be too high (above the pre-determined level) can be tested again, for example after one or two or three days, or a week, or more, and if the level of LAG-3-HA binding inhibitor in the patient samples has dropped below the pre-determined level, the patient may be administered a second or follow-on dose of LAG-3-HA binding inhibitor.

A therapeutically effective amount of a nucleic acid encoding the LAG-3-HA binding inhibitor or a therapeutically effective fragment thereof can be administered to a subject. The LAG-3-HA binding inhibitor can be expressed by attenuated viral hosts or vectors or bacterial vectors, which can be administered to a subject. Recombinant vaccinia virus, adeno-associated virus (AAV), herpes virus, retrovirus, cytomegalovirus, poxvirus or other viral vectors can be used to express the LAG-3-HA binding inhibitor. For example, vaccinia vectors are described in U.S. Pat. No. 4,722,848. BCG (Bacillus Calmette Guerin) provides a further exemplary vector which may be utilized for expression of the disclosed LAG-3-HA binding inhibitor (see Stover, Nature 351:456-460, 1991).

In one embodiment, a nucleic acid encoding the LAG-3-HA binding inhibitor or a therapeutically effective fragment thereof is introduced directly into cells. For example, the nucleic acid can be loaded onto gold microspheres by standard methods and introduced into the skin by a device such as Bio-Rad's Heliosä Gene Gun. The nucleic acids can be “naked,” consisting of plasmids under control of a strong promoter.

Typically, the DNA is injected into muscle, although it can also be injected directly into other sites. Dosages for injection are usually around 0.5 mg/kg to about 50 mg/kg, and typically are about 0.005 mg/kg to about 5 mg/kg (see, e.g., U.S. Pat. No. 5,589,466).

A therapeutically effective amount of a LAG-3-HA binding inhibitor (or the nucleic acid encoding the LAG-3-HA binding inhibitor) will depend upon the severity of the disease and/or infection and the general state of the patient's health. A therapeutically effective amount of the LAG-3-HA binding inhibitor is that which provides either subjective relief of a symptom(s) or an objectively identifiable improvement as noted by the clinician or other qualified observer. These compositions can be administered in conjunction with another therapeutic agent, either simultaneously or sequentially.

In one embodiment, administration of the LAG-3-HA binding inhibitor (or nucleic acid encoding the LAG-3-HA binding inhibitor) results in a reduction in the establishment of influenza virus infection and/or reducing subsequent disease progression in a subject. A reduction in the establishment of influenza virus infection and/or a reduction in subsequent disease progression encompass any statistically significant reduction in viral activity.

In some embodiments, methods are disclosed for treating a subject with an influenza virus infection. These methods include administering to the subject a therapeutically effective amount of a LAG-3-HA binding inhibitor, or a nucleic acid encoding the LAG-3-HA binding inhibitor, thereby preventing or treating the influenza virus infection.

In additional embodiments, the subject is also administered an effective amount of an additional agent, such as anti-viral agent. The methods can include administration of one on more additional agents known in the art. For any application, the LAG-3-HA binding inhibitor, therapeutically effective fragment thereof, or nucleic acid encoding the LAG-3-HA binding inhibitor or respective fragment, provided, e.g., as pharmaceutical compositions, can be administered either alone or in combination with one or more other therapy, e.g., the administration of a second or additional therapeutic agent. In some embodiments, the combination can result in a lower dose of the LAG-3-HA binding inhibitor or of the other therapy being needed, which, in some embodiments, can reduce side effects. In some embodiments, the combination can result in enhanced delivery or efficacy of one or both agents. The agents or therapies can be administered at the same time (e.g., as a single formulation that is administered to a patient or as two separate formulations administered concurrently) or sequentially in any order. The sequential administrations can be provided on the same day (e.g., within one hour of one another or at least 3, 6, or 12 hours apart) or on different days.

Such second or additional agents include vaccines, anti-viral agents, and/or antibodies. According to some embodiments the second or additional agent is not co-formulated with the LAG-3-HA binding inhibitor, according to other embodiments it is. In some embodiments, the LAG-3-HA binding inhibitor and the second or additional agent are administered such that one or more of the following is achieved: therapeutic levels, or therapeutic effects, of one overlap the other; detectable levels of both are present at the same time; or the therapeutic effect is greater than what would be seen in the absence of either the LAG-3-HA binding inhibitor, or the second or additional agent. In some embodiments, each agent will be administered at a dose and on a time schedule determined for that agent.

The second or additional agent can be, for example, for treatment or prevention of influenza. For example, the LAG-3-HA binding inhibitors provided herein can be administered in combination with a vaccine, e.g., a vaccine described herein or a mixture (a.k.a. a cocktail) of influenza peptides to stimulate the patient's immune system to prevent infection with particular strains of influenza A and/or B. In other examples, the second or additional agent is an anti-viral agent (e.g., an anti-NA or anti-M2 agent), a pain reliever, an anti-inflammatory, an antibiotic, a steroidal agent, an antibody molecule (e.g., an anti-HA antibody), an adjuvant, a protease or glycosidase (e.g., sialidase), etc.

Exemplary anti-viral agents include vaccines, neuraminidase inhibitors and nucleoside analogs. Exemplary anti-viral agents can include, e.g., zidovudine, gangcyclovir, vidarabine, idoxuridine, trifluridine, foscarnet, acyclovir, ribavirin, amantadine, remantidine, saquinavir, indinavir, ritonavir, alpha-interferons and other interferons, a neuraminidase inhibitor (e.g., zanamivir (Relenza®), oseltamivir (Tamiflu®), laninamivir, peramivir), rimantadine, a PB2 inhibitor (e.g., pimodivir), and an endonuclease inhibitor (e.g., the cap-dependent endonuclease inhibitor, e.g., baloxavir marboxil).

Single or multiple administrations of the compositions including the LAG-3-HA binding inhibitor, therapeutically effective fragment thereof, or nucleic acid encoding the LAG-3-HA binding inhibitor or respective fragment, that are disclosed herein, may be administered depending on the dosage and frequency as required and tolerated by the patient. In any event, the composition should provide a sufficient quantity of at least one of the LAG-3-HA binding inhibitor disclosed herein to effectively treat the subject. The dosage may be administered once or may be applied periodically until either a therapeutic result is achieved or until side effects warrant discontinuation of therapy.

According to some embodiments of the present invention, the compositions including the LAG-3-HA binding inhibitor may be an extended-release composition.

Generally, the dose is sufficient to treat or ameliorate symptoms or signs of disease without producing unacceptable toxicity to the subject.

### EXAMPLES

### Example 1: LAG-3 Binds to Influenza Virus-Infected Cells and Directly Interacts with Influenza Virus HA

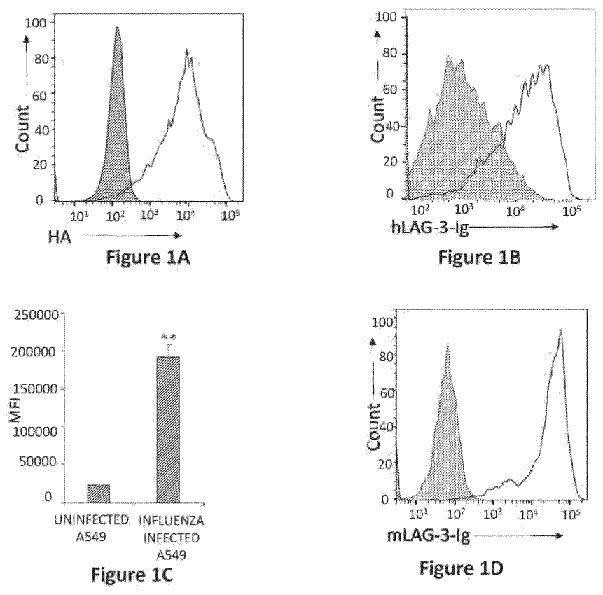

A major hurdle in modulating LAG-3 activity is the elusive nature of its ligands. As a preliminary stage to attempting to identify a particular ligand, it was first tested whether LAG-3 can recognize viral antigens or virus-infected cells. To this end, the extracellular domain of human LAG-3 protein (amino acids 23-450) (SEQ ID NO. 2) was fused with the constant region of human IgG1 (Fc domain) (SEQ ID NO. 3), creating a hLAG-3-Ig conjugate (herein also “LAG-3 conjugate”) (SEQ ID NO. 4). A549 epithelial cells were infected with influenza virus A/Puerto Rico/8/1934 (H1N1). 48 hours following infection, monoclonal anti-HA antibody was added to the cell medium in order to stain the cells and verify viral infection (FIG. 1A), and hLAG-3-Ig was added to test the interaction thereof with infected cells (FIG. 1B). The level of staining was compared to the staining of uninfected cells A549 cells with anti-HA and with hLAG-3-Ig as a negative control (FIG. 1A-B).

A significant increase in hLAG-3-Ig binding was seen in influenza virus-infected cells in comparison with uninfected cells (student's t test, **p<0.005) (FIG. 1B-C). A similar result was observed with mouse LAG-3 (mLAG-3-Ig, FIG. 1D).

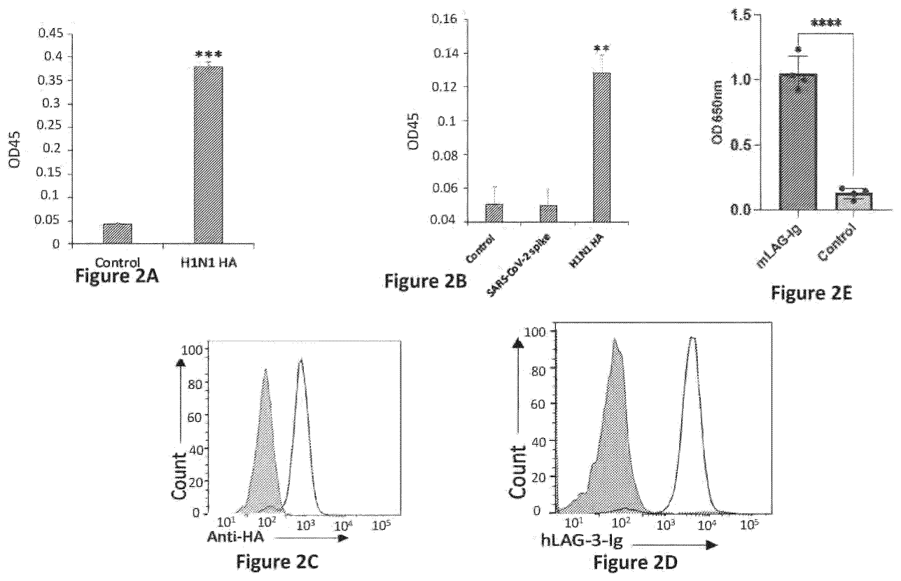

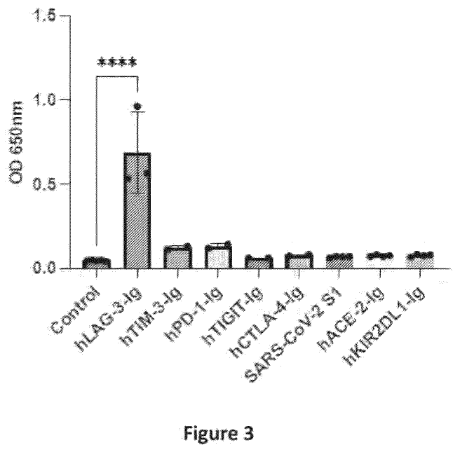

After establishing the binding of hLAG-3-Ig in influenza virus infected cells, identification of the specific ligand that is recognized by LAG-3 was pursued. The binding of hLAG-3 with the HA glycoprotein, one of the viral glycoproteins that is expressed on the external surface of infected cells, was tested using ELISA. An ELISA plate was coated with soluble LAG-3-Ig and incubated with the HA glycoprotein of A/Puerto Rico/8/1934 (H1N1) influenza virus (without the rest of the virus/a virus infected cell). Unexpectedly, direct binding of LAG-3 and HA was observed (student's t test, ***p<0.0005) (FIG. 2A). As a negative control for recognition of viral glycoproteins by LAG-3, binding of LAG-3 and the spike glycoprotein of SARS-COV-2 was tested. No binding of LAG-3 to the spike was observed (FIG. 2B). As a further negative control, to verify that the binding of HA is not by virtue of the IgG1 component of hLAG-3-Ig, additional alternative proteins (hTIM-3-Ig, hPD-1-Ig, hTIGIT-Ig, hCTLA-4-IG, SARS COV-2-S1, ACE-2, and KIR2DL1) were fused to the IgG1 Fc domain, and were tested for binding to HA using ELISA. The viral HA was shown to interact only with LAG-3-Ig and with no other protein fused to IgG1 Fc domain (student's T test, ****p<0.00005) (FIG. 3).

Next, the HA glycoprotein of influenza virus A/Puerto Rico/8/1934 (H1N1) was expressed on the surface of MDCK cells (FIGS. 2C-D), to examine the binding of hLAG-3-Ig to HA when expressed on a cell surface. Expression of HA on the surface of MDCK cells lead to increased binding of LAG-3-Ig to the cells (FIG. 2D), indicating the LAG-3 binds HA also in native conditions. A similar result was observed also when mouse LAG-3 binding was tested (FIG. 2E).

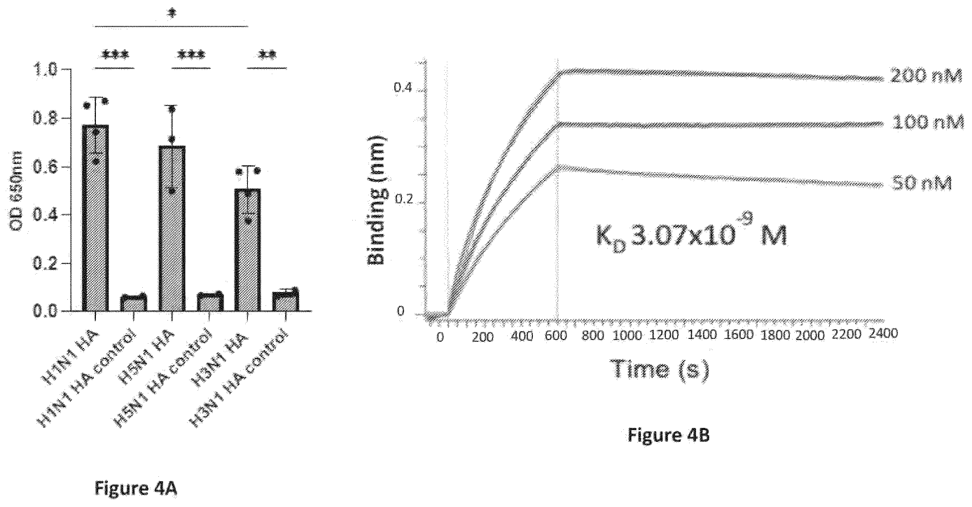

To determine whether the HA-LAG-3 binding is specific to a particular influenza strain, LAG-3 was incubated with ELISA plates coated with HA proteins from different Influenza A viral strains (i.e., displaying different HA subtypes), in addition to the originally tested HA from influenza A H1N1. The results are shown in FIG. 4A. Importantly, as can be seen, significant binding of LAG-3-HA was found for all tested Influenza strains (Influenza A H5N1 (A/Vietnam/1194/2004), Influenza A H3N2 (A/Wisconsin/67/2005)), indicating that the LAG-3 interaction with HA is not strain specific.

The Bio-layer interferometry (BLI) assay was performed using an OctetR4 instrument to study the binding affinities between the HA and human LAG-3 (ACROBiosystems). The assay was initiated by hydrating with the Octet® Anti-Penta-HIS (HIS1K) Biosensors (Sartorius, Cat. 18-5120) for 10 minutes. After hydration, BLI was performed over five steps: initial baseline, antigen immobilization, second baseline, antibody binding, and dissociation. Baseline and dissociation steps were performed using the Octet® Kinetics Buffer (Sartorius, Cat. 18-1105). First, HIS1K biosensor surface was loaded for antigen immobilization with the Polyhistidine-tagged recombinant HA. This was followed by the removal of the residual antigen in the second baseline step and loading of human LAG-3 in different concentrations (50 nM, 100 nM, 200 nM) to evaluate the specific interaction between the HA and human LAG-3. Based on the 1:1 binding model, the binding constants were calculated from the resulting association-dissociation curves. The data was analyzed using the Octet Analysis Studio.

As shown in FIG. 4B, the binding kinetics assay between human LAG-3 and HA reveals dose-dependent changes in affinity (KD).

### Example 2: The Binding of LAG-3 to Influenza Virus HA is Glycan-Dependent

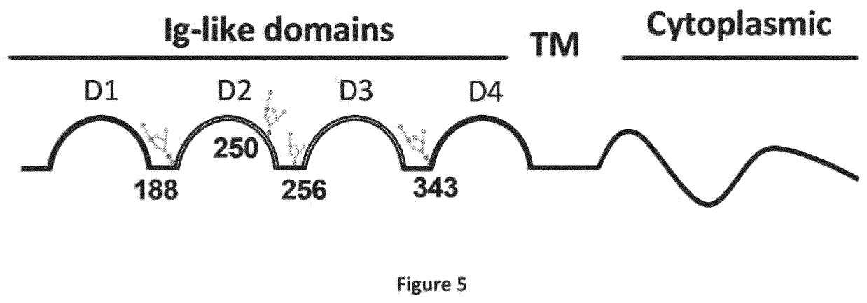

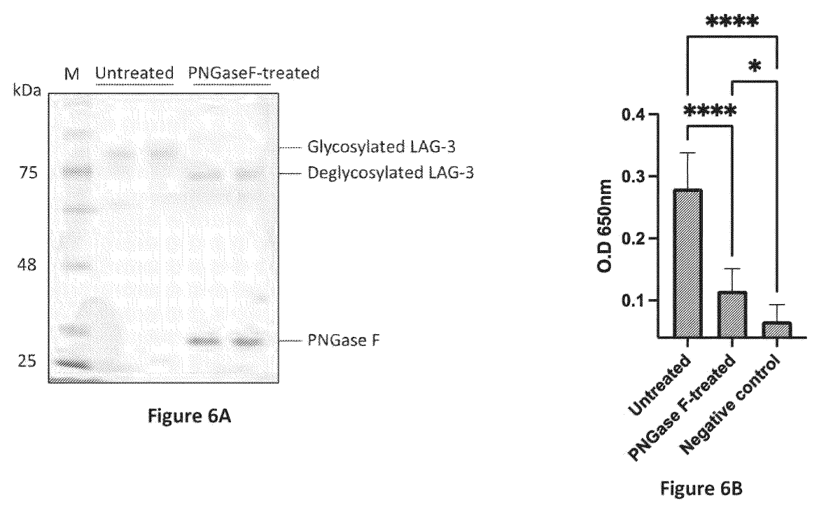

Following identification of the direct and specific binding of LAG-3 with HA, further evaluation of binding characteristics was performed. In light of the reported ability of HA to bind sialic acids residues that are present on glycosylated proteins (Bar-On et al. (2013), Cell Rep., Vol. 3 (4), 1044-1050; Bar-On et al. (2014), Journ. Infect. Diseas., Vol. 210 (3) 410-418), LAG-3-Ig was treated with PNGase F (an enzyme that removes N-linked oligosaccharides from glycoproteins) to test whether removal of the glycans from the LAG-3 N-glycosylation sites affects the interaction with HA. LAG-3 N-glycosylation sites are located at amino acids 188, 250, 256, and 343 (FIG. 5). As is shown in FIGS. 6A and 6B, the removal of N-linked glycans from LAG-3 (using PNGase F) significantly reduced the interaction of LAG-3 with HA. This established the glycan-dependence of the LAG-3-HA interaction.

### Example 3: The Binding of LAG-3 to Influenza Virus HA is Through LAG-3's D2 Domain

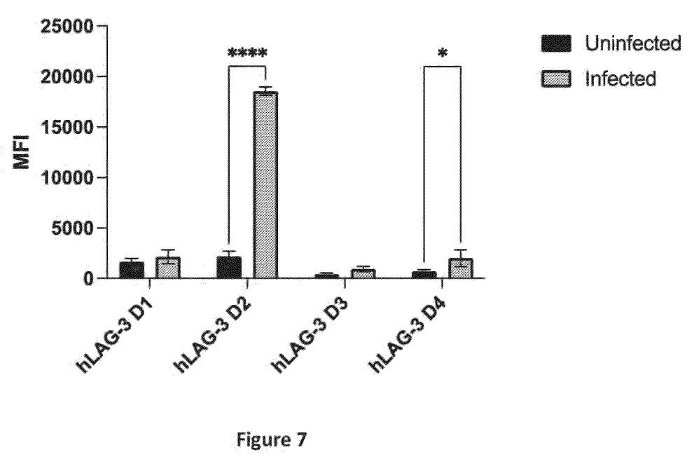

The LAG-3 protein is composed of 4 extracellular domains: D1, D2, D3 and D4 (corresponding to SEQ ID Nos. 6, 7, 8 and 9 respectively). In order to identify the binding domain in LAG-3 which is responsible for the binding of HA, each one of these domains were fused to human IgG1 Fc region. Next, these fusion proteins were used to stain uninfected and influenza virus infected A549 cells. As can be seen in FIG. 7, a significant binding of the D2 domain to infected cells, a minor binding of the D4 domain and no binding of the other domains was observed. This indicates the D2 domain of human LAG-3 is responsible for binding the influenza HA. These results also coincide with the previous observation that the binding of LAG-3 and HA is glycan-dependent, since the D2 domain contains 3 N-linked glycosylation sites.

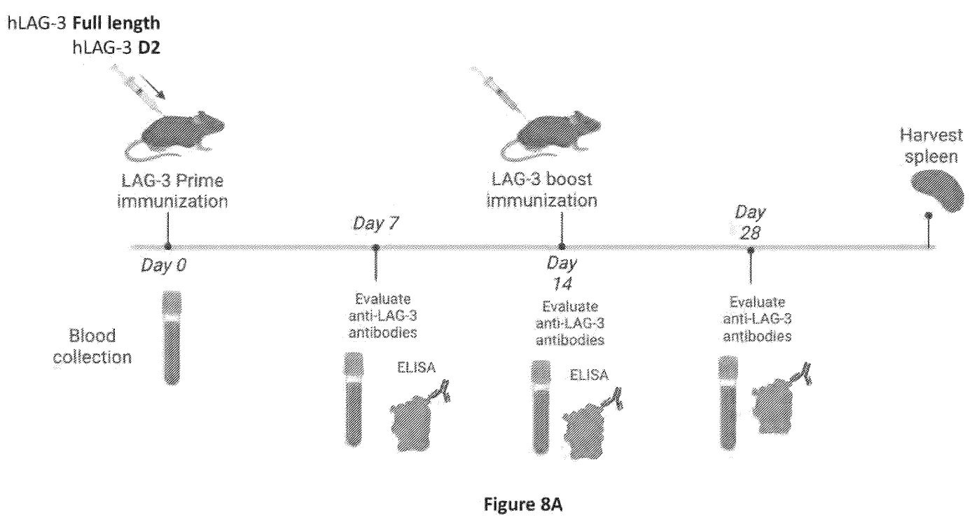

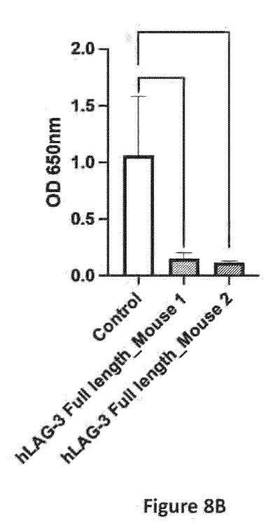

After revealing the binding site of human LAG-3 with HA, C57BL/6 mice were immunized with human LAG-3 or with the human LAG-3 D2 domain (SEQ ID NO. 6) by injecting the protein with an adjuvant three time (intraperitoneal injection) (FIG. 8A). The elicitation of antibodies that recognize LAG-3 was verified by bleeding the mice and testing the serum binding to human LAG-3 by ELISA. ELISA plates were coated with LAG-3D2-Fc protein, then incubated with serum from mice immunized with hLAG-3 (polyclonal antibodies). After washing, influenza virus HA protein was added to each well. The interaction was evaluated using an HRP-conjugated secondary anti-His Tag antibody. The absorbance was quantified at 650 nm after the addition of TMB (3,3′,5,5′-tetramethylbenzidine) substrate. Importantly, as can be seen in FIG. 8B, polyclonal antibodies that were elicited in the immunized mice can bind to human LAG-3 D2 domain and block the interaction between human LAG-3 D2 domain and HA.

Spleens from the immunized mice were harvested, B cells were sorted at a single-cell level to produce monoclonal antibodies. The antibodies that are produced by these T cells were sequenced. The clone names as well as the SEQ ID Nos. in the Sequence Listing of the CDR sequences and light chain (LC) and heavy chain (HC) variable region (VR) sequences are presented in Table 1.

TABLE 1
Monoclonal antibodies identified, cloned and sequences.

| | | | | Nucleotides | |
|---|---|---|---|---|---|
| Antibody | Amino acids (SEQ ID NO.) | | | (SEQ ID NO) | |
| name | LC-VR | HC-VR | CDRs | LC-VR | HC-VR |
| 23DKTYB4* | 9 | 10 | 35, YTS, 36, 37, 38, 39 | 81 | 82 |
| 23DKTYB10* | 11 | 12 | 35, YTS, 40, 41, 42, 43 | 83 | 84 |
| 23DKTYB11* | 13 | 12 | 35, YTS, 44, 41, 42, 43 | 85 | 84 |
| 23DKTYB13* | 14 | 12 | 45, AAT, 46, 41, 42, 43 | 86 | 84 |
| 23DKTYB14* | 15 | 12 | 47, STS, 48, 41, 42, 43 | 87 | 84 |
| 23DKTYB15* | 15 | 16 | 47, STS, 48, 41, 42, 49 | 87 | 88 |
| 23DKTYB18* | 17 | 12 | 50, AAS, 51, 41, 42, 43 | 89 | 84 |
| 23DKTYB19* | 17 | 18 | 50, AAS, 51, 37, 38, 52 | 89 | 90 |
| 23DKTYB20* | 19 | 12 | 53, SAS, 54, 41, 42, 43 | 91 | 84 |
| 23DKTYB21* | 19 | 20 | 53, SAS, 54, 76, 55, 56 | 91 | 92 |
| 23DKTYB1 | 21 | 12 | 57, WTS, 58, 41, 42, 43 | 93 | 84 |
| 23DKTYB2 | 22 | 12 | 59, KVS, 60, 41, 42, 43 | 94 | 84 |
| 23DKTYB3 | 9 | 12 | 35, YTS, 36, 41, 42, 43 | 81 | 84 |
| 23DKTYB5 | 23 | 12 | 45, AAT, 61, 41, 42, 43 | 95 | 84 |
| 23DKTYB6 | 24 | 12 | 62, LVS, 63, 41, 42, 43 | 96 | 84 |
| 23DKTYB7 | 24 | 25 | 62, LVS, 63, 64, 65, 66 | 96 | 97 |
| 23DKTYB8 | 26 | 12 | 67, RCT, 68, 41, 42, 43 | 98 | 84 |
| 23DKTYB9 | 26 | 27 | 67, RCT, 68, 41, 42, 43 | 98 | 99 |
| 23DKTYB12 | 28 | 12 | 45, AAT, 69, 41, 42, 43 | 100 | 84 |
| 23DKTYB16 | 29 | 12 | 70, AAS, 71, 41, 42, 43 | 101 | 84 |
| 23DKTYB17 | 29 | 30 | 70, AAS, 71, 41, 72, 73 | 101 | 102 |
| 23DKTYB22 | 31 | 12 | 74, DTS, 75, 41, 42, 43 | 103 | 84 |
| 23DKTYB23 | 31 | 32 | 74, DTS, 75, 76, 77, 78 | 103 | 104 |
| 23DKTYB24 | 33 | 12 | 59, KVS, 79, 41, 42, 43 | 106 | 84 |
| 23DKTYB25 | 34 | 12 | 62, LVS, 80, 41, 42, 43 | 105 | 84 |

*These monoclonal antibodies bind specifically to the D2 domain of human LAG-3.

### Example 4: Influenza Virus HA Glycoprotein Impairs T-Cell Activation Ex Vivo

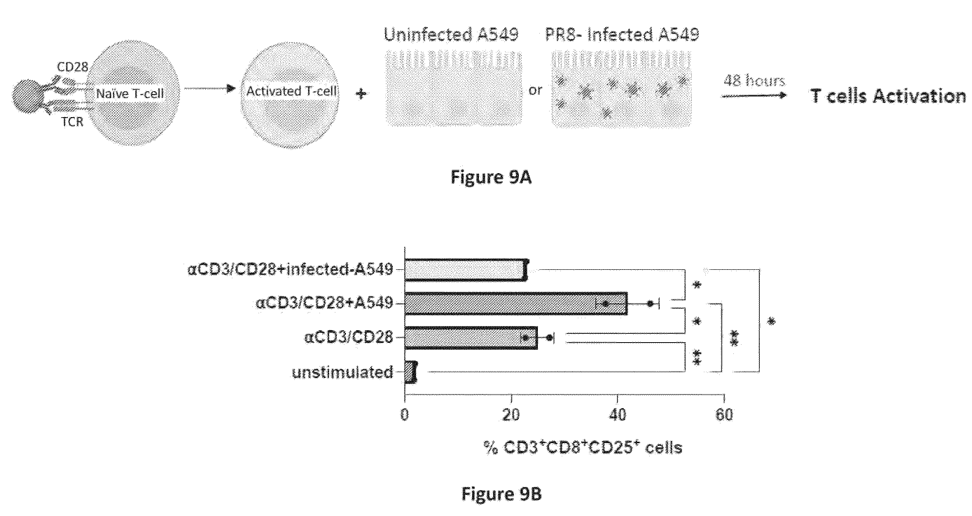

In order to test the effect of influenza virus HA glycoprotein on T-cell activation, mouse T cells were activated by CD3 and CD28 beads and added to uninfected or infected A549 cells. After 48 hours the activation markers of T cells were monitored using flow cytometry (FIG. 9A). As can be seen in FIG. 9B, influenza virus HA glycoprotein impaired T-cell activation, which was monitored by the percentage of CD8+CD25+ cells.

### Example 5: MLAG-3 Expression on T-Cell Increases Post Influenza Infection In Vivo

C57BL/6 mice were infected with H1N1 influenza virus (40 PFU) and were untreated or treated with LAG3-Ig. Mice weight loss was monitored every day following infection, mice lungs were harvested on days 3, 5, 7 and 10 post infection, and the % of LAG-3 positive T cells and the levels of LAG-3 expression of CD8+ T cells were evaluated in untreated and LAG-3-Ig-treated mice.

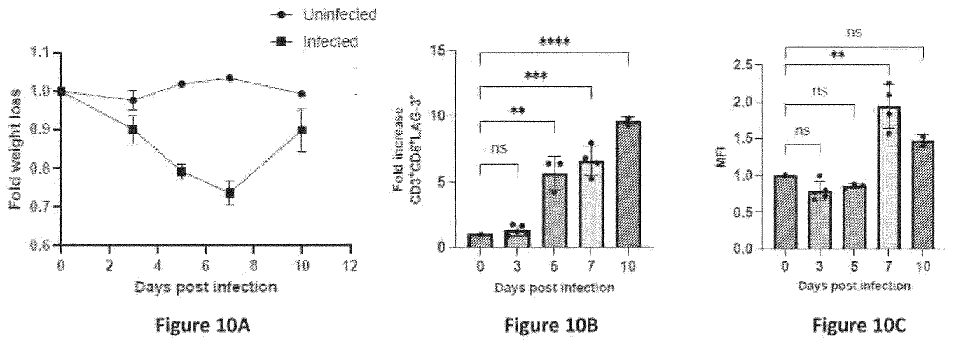

As can be seen in FIG. 10A, the flu-infected untreated mice showed a significant weight loss, especially on day 7 from infection. FIG. 10B displays a sharp increase in the percentage of LAG-3-positive CD8+ T cells in the lungs of infected mice, starting at 5 days post infection (DPI) and peaking at 10 DPI. FIG. 10C presents the increase in surface expression level of LAG-3 on CD8+ T cells that were isolated from the infected mice at 7 DPI, similar to the weight loss [negative] peak observed in these mice.

### Example 6: Inhibition of HA-LAG-3 Interaction Increases T-Cell Activation In Vivo

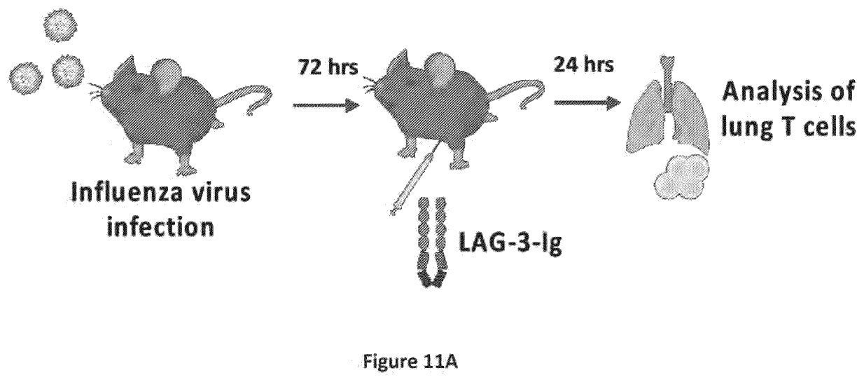

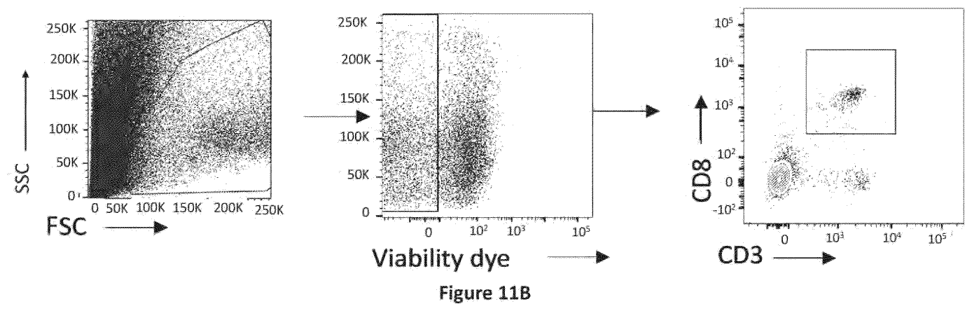

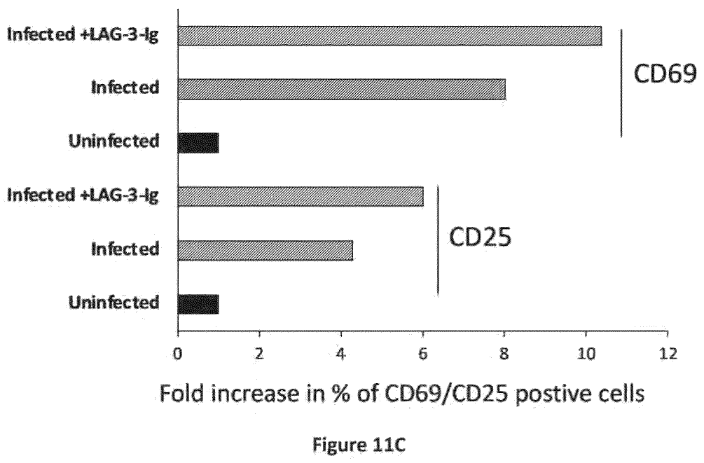

In view of the known inhibitory and T cell suppression function of LAG-3, it was tested whether impairing the interactions between HA and LAG-3 during influenza virus infection in vivo would influence the T cell responses toward the infected cells. C57BL/6 mice were divided into three groups of 6 mice each; two groups were infected with X MOI of A/Puerto Rico/8/1934 (H1N1), and one control uninfected group. 3 days post infection one group received LAG-3-Ig (as a LAG-3-HA binding inhibitor) injected intraperitoneally, while the other group of infected mice remained untreated. In order to dissect only the effects of the impaired HA-LAG-3 interaction induced by LAG-3-Ig, LAG-3 was fused to a human IgG1 Fc constant region that does not activate antibody-dependent cellular cytotoxicity (ADCC) in mouse immune cells. 24 hours after the LAG-3-Ig injections, the lungs of all the mice were harvested and the levels of lung CD8⁺ T cells activation was evaluated by staining the cells to the activation markers CD69 and CD25 (FIG. 11A). As expected, increased percentage of CD69⁺CD8⁺ and CD25⁺CD8⁺ cells were seen in the lungs following infection, as compared to uninfected mice (control group). Of note, this percentage was further increased in the lungs of the infected mice that were treated with LAG-3-Ig (FIG. 11B-C).

Thus, LAG-3-Ig treatment during influenza virus infection augmented the activity of lung CD8⁺ T cells, reducing the inhibitory effect of LAG-3-Ig on LAG-3-HA binding.

### Example 7: Testing the Therapeutic Potential of LAG-3-Ig

Following the observation that the LAG-3-HA inhibitor hLAG-3-Ig enhanced T cell activity in infected mice, the therapeutic effects of hLAG-3-Ig on Influenza infected mice is tested. High amount of the LAG-3-Ig polypeptide, for in vivo experiments, is produced by well-known expression systems, e.g., the ExpiCHO system (Jain et al. (2017), Protein Expr Purif., 134). In order to dissect only the effects of the impaired HA-LAG-3 interaction induced by LAG-3-Ig, LAG-3 is fused to a mutated version of mouse IgG1 Fc constant region (D265A) that does not bind Fc receptors on mouse immune cells which activate antibody-dependent cellular cytotoxicity (ADCC) (Baudino et al. (2008), Journal of Immun., 181 (9)).

In a typical in vivo experiment, C57BL/6 mice are infected with 100 MOI of A/Puerto Rico/8/1934 (H1N1) influenza virus, and three days following the infection are treated with respective doses of the LAG-3-Ig (0 (control), 2 mg/kg, 6 mg/kg, 15 mg/kg). Mice weight loss, survival, lung viral loads, and lung T cell activation state are analyzed. In addition, the ability of LAG-3-Ig to prevent infection in a prophylaxis setting is also tested. For the prevention chart, the fusion proteins are first injected to the mice, and 24 hours later the mice are infected with X MOI of A/Puerto Rico/8/1934 (H1N1) influenza virus.

In this experiment the fusion protein is injected intraperitoneally and the levels of the LAG-3-Ig in the circulation are monitored by collecting blood samples and preforming direct ELISA for detection of LAG-3 in the plasma. In following experiments, other routs of administration, including IV administration, inhalation and nasal spraying, are tested.

### Example 8: Development of Monoclonal Antibodies that Block the HA-LAG-3 Interaction, and Assessment of their Therapeutic Potential

To generate specific and effective monoclonal antibodies (mAbs) (Freund et al. (2017), Sci Transl Med., 9 (373)), mice are immunized with the extracellular domain of human LAG-3 immunogens and the spleen of the immunized mice is used for single-cell sorting of antigen-specific memory B cells and cloning of the antibodies they encode (von Boehmer et al. (2016), Nat Protoc., 11 (10)). The different antibodies that are cloned, are then screened for their ability to block the binding of the human LAG-3 with the influenza HA. This is tested by an ELISA assay in which ELISA plates are coated with influenza virus HA, and the binding of the human LAG-3 to the HA is tested, respectively, with and without prior incubation of the human LAG-3 with the cloned antibodies. Antibodies which are found to impair the binding of HA and human LAG-3, are further tested for their ability to block this interaction in the context of infected cells, using flow cytometry. Influenza virus infected cells (A549 cells) which bind LAG-3-Ig are stained, and the effectivity of the different antibodies in blocking LAG-3-Ig binding to the infected cells is quantified according to the reduction in cell staining.

The mAbs ability to effect T-cell activation is also tested; Mouse or human T cells are activated and then exposed to the Influenza virus Hemagglutinin protein, with or without the addition of anti-LAG-3 monoclonal antibody (mAb). After 48 hours of incubation, the activation markers of T cells are monitored using flow cytometry (specifically CD69) and ELISA (measuring Perforin, Granzyme B, and Interferon gamma levels).

Antibodies which are found to block HA-LAG-3 interactions are further tested to identify those that specifically impair the interaction of LAG-3 with HA, without altering the binding of other ligands of LAG-3, such as MHC-II.

The antibodies which display the most effective blocking of the HA-LAG-3 binding in the ELISA and flow cytometry analyses, are further examined for their ability to inhibit HA-LAG-3 interaction using, for example, recombinant Jurkat T cells expressing firefly luciferase gene, under the control of NFAT response elements (Wang et al. (2020), Luminescence., 35 (8) with constitutive expression of human LAG-3. The LAG-3 expressing Jurkat cells are incubated with influenza virus infected cells, and the levels of T cell activation are tested by comparing the luciferase expression in the Jurkat cells with or without the presence of the different LAG-3 antibodies. To evaluate the binding affinity of monoclonal mouse anti-LAG-3 antibodies to LAG-3 protein, ELISA and flow cytometry techniques are employed. In the ELISA assay, ELISA plates are coated with human or mouse LAG-3 protein overnight. Subsequently, after washing and blocking, monoclonal mouse anti-LAG-3 antibodies are added to the wells, followed by incubation with an HRP-conjugated secondary anti-mouse antibody. The absorbance is then measured at 650 nm after the addition of TMB substrate. For flow cytometry analysis, activated CD8+ T-cells are stained with the monoclonal mouse anti-LAG-3 antibodies followed by a conjugated secondary anti-mouse antibody. The fluorescence emitted by the stained cells is then measured using flow cytometry.

To assess the inhibitory effect of anti-LAG-3 antibodies on LAG-3 and HA interaction, similar ELISA and flow cytometry techniques are employed. In the ELISA assay, ELISA plates are coated with LAG-3 protein, followed by incubation with the monoclonal mouse anti-LAG-3 antibodies. After washing, HA protein from different influenza virus strains is added to each well, and the interaction is assessed using an HRP-conjugated secondary anti-His Tag antibody. The absorbance is measured at 650 nm after the addition of TMB substrate. For flow cytometry analysis, monoclonal mouse anti-LAG-3 antibodies are first incubated with LAG-3-Fc protein and then used to stain MDCK-HA expressing cells or A549 PR8 infected cells. The fluorescence emitted by the stained cells is measured using flow cytometry after incubation with a labeled secondary anti-human antibody.

At least one of the most potent human LAG-3 antibodies is chosen, following the luciferase assay, for further evaluations of its ability to assist in prevention, amelioration or treatment of Influenza virus infection. The further evaluation includes, for example, the processes described in Experiment 6 and Experiment 7 above, where the LAG-3 antibody is used in place of LAG-3-Ig. Another evaluation includes infecting mice with a lethal dose of Influenza virus and then treating with either 2 mg or 4 mg of anti-LAG-3 mAb two days after infection. The weight loss and survival of the mice is then tracked for a period of 14 days and compared to infected mice that did not receive treatment.

The ability of the anti-LAG-3 antibodies to interact with mammalian ligands of LAG-3 is tested by any method know in the art. For example, by immunoassay such as ELISA. Animal models known in the art are used to test the therapeutic potential of the antibodies against cancer, autoimmunity, and CNS disorders.

1. An antibody, or an antibody fragment thereof comprising at least the antigen binding portion, which specifically binds to an N-glycosylation site within residues 169-351 of human LAG-3 (SEQ ID NO. 1), said antibody or fragment thereof comprises a set of six complementarity determining region (CDR) sequences, 3 of a light chain variable region (LC-VR) and 3 of a heavy chain variable region (HC-VR), wherein the set is selected from the group consisting of:
i. SEQ ID No. 35, YTS, SEQ ID No. 36, SEQ ID No. 37, SEQ ID No. 38, and SEQ ID No. 39;
ii. SEQ ID No. 35, YTS, SEQ ID No. 40, SEQ ID No. 41, SEQ ID No. 42, and SEQ ID No. 43;
iii. SEQ ID No. 35, YTS, SEQ ID No. 44, SEQ ID No. 41, SEQ ID No. 42, and SEQ ID No. 43;
iv. SEQ ID No. 45, AAT, SEQ ID No. 46, SEQ ID No. 41, SEQ ID No. 42, and SEQ ID No. 43;
v. SEQ ID No. 47, STS, SEQ ID No. 48, SEQ ID No. 41, SEQ ID No. 42, and SEQ ID No. 43;
vi. SEQ ID No., STS, SEQ ID No. 48, SEQ ID No. 41, SEQ ID No. 42, and SEQ ID No. 49;
vii. SEQ ID No. 50, AAS, SEQ ID No. 51, SEQ ID No. 41, SEQ ID No. 42, and SEQ ID No. 43;
viii. SEQ ID No. 50, AAS, SEQ ID No. 51, SEQ ID No. 37, SEQ ID No. 38, and SEQ ID No. 52;
ix. SEQ ID No. 53, SAS, SEQ ID No. 54, SEQ ID No. 41, SEQ ID No. 42, and SEQ ID No. 43;
x. SEQ ID No. 53, SAS, SEQ ID No. 54, SEQ ID No. 76, SEQ ID No. 55, and SEQ ID No. 56;
xi. SEQ ID No. 57, WTS, SEQ ID No. 58, SEQ ID No. 41, SEQ ID No. 42, and SEQ ID No. 43;
xii. SEQ ID No. 59, KVS, SEQ ID No. 60, SEQ ID No. 41, SEQ ID No. 42, and SEQ ID No. 43;
xiii. SEQ ID No. 35, YTS, SEQ ID No. 36, SEQ ID No. 41, SEQ ID No. 42, and SEQ ID No. 43;
xiv. SEQ ID No. 45, AAT, SEQ ID No. 61, SEQ ID No. 41, SEQ ID No. 42, and SEQ ID No. 43;
XV. SEQ ID No. 62, LVS, SEQ ID No. 63, SEQ ID No. 41, SEQ ID No. 42, and SEQ ID No. 43;
xvi. SEQ ID No. 62, LVS, SEQ ID No. 63, SEQ ID No. 64, SEQ ID No. 65, and SEQ ID No. 66;
xvii. SEQ ID No. 67, RCT, SEQ ID No. 68, SEQ ID No. 41, SEQ ID No. 42, and SEQ ID No. 43;
xviii. SEQ ID No. 45, AAT, SEQ ID No. 69, SEQ ID No. 41, SEQ ID No. 42, and SEQ ID No. 43;
xix. SEQ ID No. 70, AAS, SEQ ID No. 71, SEQ ID No. 41, SEQ ID No. 42, and SEQ ID No. 43;
XX. SEQ ID No. 70, AAS, SEQ ID No. 71, SEQ ID No. 41, SEQ ID No. 72, and SEQ ID No. 73;
xxi. SEQ ID No. 74, DTS, SEQ ID No. 75, SEQ ID No. 41, SEQ ID No. 42, and SEQ ID No. 43;
xxii. SEQ ID No. 74, DTS, SEQ ID No. 75, SEQ ID No. 76, SEQ ID No. 77, and SEQ ID No. 78;
xxiii. SEQ ID No. 59, KVS, SEQ ID No. 79, SEQ ID No. 41, SEQ ID No. 42, and SEQ ID No. 43;
xxiv. SEQ ID No. 62, LVS, SEQ ID No. 80, SEQ ID No. 41, SEQ ID No. 42, and SEQ ID No. 43.

2. The antibody or antibody fragment according to claim 1 comprising a combination of a LC-VR and a HC-VR, wherein the combination is selected from:
i. SEQ ID Nos. 9 and 10;
ii. SEQ ID Nos. 11 and 12;
iii. SEQ ID Nos. 13 and 12;
iv. SEQ ID Nos. 14 and 12;
v. SEQ ID Nos. 15 and 12;
vi. SEQ ID Nos. 15 and 16;
vii. SEQ ID Nos. 17 and 12;
viii. SEQ ID Nos. 17 and 18;
ix. SEQ ID Nos. 19 and 12;
x. SEQ ID Nos. 19 and 20;
xi. SEQ ID Nos. 21 and 12;
xii. SEQ ID Nos. 22 and 12;
xiii. SEQ ID Nos. 9 and 12;
xiv. SEQ ID Nos. 23 and 12;
XV. SEQ ID Nos. 24 and 12;
xvi. SEQ ID Nos. 24 and 25;
xvii. SEQ ID Nos. 26 and 12;
xviii. SEQ ID Nos. 26 and 27;
xix. SEQ ID Nos. 28 and 12;
XX. SEQ ID Nos. 29 and 12;
xxi. SEQ ID Nos. 29 and 30;
xxii. SEQ ID Nos. 31 and 12;
xxiii. SEQ ID Nos. 31 and 32;
xxiv. SEQ ID Nos. 33 and 12;
XXV. SEQ ID Nos. 34 and 12;
or an analog having at least 90% sequence identity with the any of the LC-VR and HC-VR sequences.

3. The antibody or antibody fragment according to claim 1, wherein the antibody or antibody fragment is configured to bind an epitope within the D2 domain of human LAG-3 (SEQ ID NO. 6) and comprises a combination LC-VR and HC-VR selected from:
i. SEQ ID Nos. 9 and 10;
ii. SEQ ID Nos. 11 and 12;
iii. SEQ ID Nos. 13 and 12;
iv. SEQ ID Nos. 14 and 12;
v. SEQ ID Nos. 15 and 12;
vi. SEQ ID Nos. 15 and 16;
vii. SEQ ID Nos. 17 and 12;
viii. SEQ ID Nos. 17 and 18;
ix. SEQ ID Nos. 19 and 12;
x. SEQ ID Nos. 19 and 20.

4. The antibody or antibody fragment according to any one of claim 1, comprising a HC-VR comprising SEQ ID No. 12 and a LC-VR comprising a sequence selected from SEQ ID Nos. 11, 13, 14, 15, 17, 19, 21, 22, 9, 23, 24, 26, 28, 29, 33 and 34.

5. (canceled)

6. The antibody or antibody fragment according to claim 1, capable of inhibiting binding of human LAG-3 to at least one molecule selected from: human Galectin-3 (LGALS3), human C-type lectin domain family 4 member G (LSECtin) protein, human MHC class II molecules, human Fibrinogen-like protein 1 (FGL1), and human Alpha-synuclein pff (α-Syn PFF).

7. The antibody or antibody fragment according to claim 1, capable of inhibiting glycan-dependent binding of human LAG-3 to influenza hemagglutinin (HA).

8. The antibody or antibody fragment according to claim 1, wherein the glycan-dependent binding involves at least one LAG-3 N-glycosylation site located on an amino acid selected from the group consisting of: amino acid 188, amino acid 250, amino acid 256, and amino acid 343, of SEQ ID NO. 1.

9. A pharmaceutical composition comprising an antibody or antibody fragment according to claim 1, and at least one pharmaceutical acceptable excipient, diluent, salt, or carrier.

10-12. (canceled)

13. A polynucleotide encoding at least one of: a LC-VR, wherein the polynucleotide comprises a sequence selected from the group consisting of SEQ ID Nos. 81, 83, 85, 86, 87, 89, 91, 93, 94, 95, 96, 98, 100, 101, 103, 105, 106, and an analog or derivative of any one of said sequences having at least 90% identity therewith; and
a HC-VR, wherein the polynucleotide comprises a sequence selected from the group consisting of SEQ ID Nos. 82, 84, 88, 90, 92, 97, 99, 102, 104, and an analog or derivative of any one of said sequences having at least 90% identity therewith.

14-15. (canceled)

16. A method of preventing, attenuating, or treating a disease selected from cancer, autoimmune disease, inflammatory disease and a CNS disease, comprising administering to a subject at risk of or suffering from said disease or disorder, a therapeutically effective amount of an antibody or antibody fragment according to claim 1.

17. The method of claim 16, wherein the disease is cancer.

18-30. (canceled)

31. A method of preventing, attenuating, or treating a disease or disorder caused by Influenza virus, comprising administering to a subject at risk of or suffering from said disease or disorder, a therapeutically effective amount of an antibody or antibody fragment according to claim 1.

32. The method according to claim 31, wherein the Influenza virus comprises at least one of influenza A H1N1, influenza A H5N1, and influenza A H3N2.

33-44. (canceled)

45. A fusion protein comprising a mammalian LAG-3 fragment comprising at least one N-glycosylation site and a carrier protein.

46. The fusion protein of claim 45, wherein the mammalian LAG-3 fragment is a fragment of human LAG-3 having a sequence set forth in SEQ ID NO. 2.

47. (canceled)

48. The fusion protein according to claim 46 comprising SEQ ID NO. 4.

49. A method of preventing, attenuating, or treating a disease or disorder caused by an influenza virus, the method comprising administering to a subject at risk of or suffering from said disease or disorder, a therapeutically effective amount of a molecule that specifically inhibits the glycan-dependent binding of influenza hemagglutinin (HA) to mammalian Lymphocyte activation gene 3 (LAG-3), wherein the molecule comprises the fusion protein according to claim 46.

50. The method according to claim 49, wherein the Influenza virus comprises at least one of influenza A H1N1, influenza A H5N1, and influenza A H3N2.

51. A method of preventing, attenuating, or treating a disease or disorder caused by an influenza virus, the method comprising administering to a subject at risk of or suffering from said disease or disorder, a therapeutically effective amount of a molecule that specifically inhibits the glycan-dependent binding of influenza hemagglutinin (HA) to mammalian Lymphocyte activation gene 3 (LAG-3) by binding to influenza HA or by binding to LAG-3, wherein the glycan-dependent binding involves at least one LAG-3 N-glycosylation site.

52. The method according to claim 51, wherein the at least one LAG-3 N-glycosylation site is located on an amino acid selected from the group consisting of: amino acid 188, amino acid 250, amino acid 256, and amino acid 343, of SEQ ID NO. 1.
